SEQUENCE LISTING (51 sequences)

SEQ ID NO: 1 - protein, 586 aa, Yarrowia lipolytica
MSTKHLFNETDELVLKSLEGVQASRSASILSHRFKVLYNGTHSADRVAVLSGGGSGHEPAHAGFVGDNMLTGAICGPVFASPSAKQVEAGCKLVPSDKGHILVVTNYTGDMLHFGLAAEKLKSQGHKVGIIKSADDVAVDRKSGGLVGRRGLAGTVLLDKIVGGAAWDKLSFDECMAIGTEVAENTATASIGLDYCHVPGRSVENHVSLDQNECQFGLGIHNEPGVKTINPVPAPESMVDTLLKYLVSQDDPERSFVKFKEGDEVILLANNLGGISTIEMRAAVQLAREQLEKTHKIKSVRVLCGTFMSSLNAPGFSITLVNLSNGSHSKNVLKYLDAVSDAPAWVNVAPPTSVKPFINEDKIFDDETSNIKAPTLDIPEQTVVAALTQASQNIIKAEPQLTAWDTEMGDGDCGHTIEHGCRALLEYLNKNKSDPKALEIIPIVRAVVHITEEDMGGTLGAIFGIFFASFLNALLLDPLSHKTDVNVTDKLVNAANTGLESLMNHTPARPGDRTVMDVLIPYVQSLVSTKDIKEAALKAKQAAEGTKKIKPRLGRAVYVGEKDGELPPDPGAWAVYELVDGFANHK

SEQ ID NO: 2 - protein, 588 aa, Artificial
  Yarrowia EYK consensus sequence
  VARIANT (12)..(12): Replace = Asp
  VARIANT (15)..(15): Replace = Val
  VARIANT (19)..(19): Replace = Lys
  VARIANT (26)..(26): Replace = Gln
  VARIANT (32)..(32): Replace = Ser or Asn
  VARIANT (41)..(41): Replace = Leu, Asn or Thr
  VARIANT (43)..(43): Replace = Thr
  VARIANT (44)..(44): Replace = Ser or Lys
  VARIANT (45)..(45): Replace = Glu
  VARIANT (123)..(123): Replace = Ala
  VARIANT (127)..(127): Replace = Asn
  VARIANT (141)..(141): Replace = Lys
  VARIANT (156)..(156): Replace = Ile
  VARIANT (164)..(164): Replace = Ala
  VARIANT (167)..(167): Replace = Phe
  VARIANT (168)..(168): Replace = Ala
  VARIANT (173)..(173): Replace = Glu
  VARIANT (177)..(177): Replace = Glu
  VARIANT (180)..(180): Replace = Ser
  VARIANT (181)..(181): Replace = Tyr
  VARIANT (183)..(183): Replace = Ser
  VARIANT (184)..(184): Replace = Asp
  VARIANT (195)..(195): Replace = Phe
  VARIANT (205)..(205): Replace = Ser or Lys
  VARIANT (207)..(207): Replace = Ser
  VARIANT (210)..(210): Replace = Gly, Val or Asp
  VARIANT (212)..(212): Replace = Asp or Asn
  VARIANT (229)..(229): Replace = Leu or Ile
  VARIANT (230)..(230): Replace = Ser
  VARIANT (232)..(232): Replace = Ile
  VARIANT (234)..(234): Replace = Asn or Ser
  VARIANT (236)..(236): Replace = Asp
  VARIANT (237)..(237): Replace = Lys or Thr
  VARIANT (238)..(238): Replace = Leu
  VARIANT (239)..(239): Replace = Ile
  VARIANT (240)..(240): Replace = Arg or Asp
  VARIANT (244)..(244): Replace = Ser, Gln or Asp
  VARIANT (246)..(246): Replace = Ile
  VARIANT (247)..(247): Replace = Leu
  VARIANT (252)..(252): Replace = Ala, His or Pro
  VARIANT (253)..(253): Replace = Gln
  VARIANT (261)..(261): Replace = Gln
  VARIANT (262)..(262): Replace = Asn
  VARIANT (277)..(277): Replace = Val
  VARIANT (285)..(285): Replace = Leu
  VARIANT (288)..(288): Replace = Lys or Val
  VARIANT (293)..(293): Replace = Asn
  VARIANT (295)..(295): Replace = Tyr
  VARIANT (296)..(296): Replace = Asn
  VARIANT (298)..(298): Replace = Ala
  VARIANT (299)..(299): Replace = Ala or Pro
  VARIANT (307)..(307): Replace = Tyr
  VARIANT (318)..(318): Replace = Leu
  VARIANT (327)..(327): Replace = Thr
  VARIANT (329)..(329): Replace = Gln
  VARIANT (330)..(330): Replace = Asn
  VARIANT (331)..(331): Replace = Ser, Gln or Lys
  VARIANT (334)..(334): Replace = Glu or Gln
  VARIANT (335)..(335): Replace = His
  VARIANT (339)..(339): Replace = Thr
  VARIANT (340)..(340): Replace = Cys
  VARIANT (348)..(348): Replace = Val or Ile
  VARIANT (349)..(349): Replace = Ala
  VARIANT (350)..(350): Replace = Ser
  VARIANT (351)..(351): Replace = Pro
  VARIANT (352)..(352): Replace = Val
  VARIANT (353)..(353): Replace = nothing
  VARIANT (359)..(359): Replace = Ile
  VARIANT (360)..(360): Replace = Asp or Asn
  VARIANT (362)..(362): Replace = Glu
  VARIANT (363)..(363): Replace = Cys or Thr
  VARIANT (367)..(367): Replace = Glu
  VARIANT (368)..(368): Replace = Ser or Asp
  VARIANT (369)..(369): Replace = Tyr, Ala or Ile
  VARIANT (370)..(370): Replace = nothing
  VARIANT (372)..(372): Replace = Gln or Thr
  VARIANT (373)..(373): Replace = Val or Leu
  VARIANT (374)..(374): Replace = Ile
  VARIANT (375)..(375): Replace = Glu
  VARIANT (377)..(377): Replace = Asn or Thr
  VARIANT (378)..(378): Replace = Ile
  VARIANT (379)..(379): Replace = Pro
  VARIANT (380)..(380): Replace = Ile
  VARIANT (381)..(381): Replace = Asp or Ser
  VARIANT (382)..(382): Replace = Gln or Thr
  VARIANT (383)..(383): Replace = Thr or Lys
  VARIANT (384)..(384): Replace = Glu
  VARIANT (385)..(385): Replace = Leu or Val
  VARIANT (386)..(386): Replace = Val
  VARIANT (387)..(387): Replace = Ser
  VARIANT (390)..(390): Replace = Lys
  VARIANT (394)..(394): Replace = Ala or Glu
  VARIANT (397)..(397): Replace = Val
  VARIANT (405)..(405): Replace = Glu
  VARIANT (424)..(424): Replace = Lys
  VARIANT (427)..(427): Replace = Val
  VARIANT (428)..(428): Replace = Lys or Ser
  VARIANT (431)..(431): Replace = Glu, Asp or His
  VARIANT (432)..(432): Replace = Asn
  VARIANT (434)..(434): Replace = Gln
  VARIANT (435)..(435): Replace = Gly or Asn
  VARIANT (436)..(436): Replace = Asn
  VARIANT (437)..(437): Replace = Ser or Thr
  VARIANT (438)..(438): Replace = Glu or Thr
  VARIANT (441)..(441): Replace = Lys
  VARIANT (448)..(448): Replace = Glu or Asp
  VARIANT (449)..(449): Replace = Ile
  VARIANT (478)..(478): Replace = His or Ser
  VARIANT (480)..(480): Replace = His
  VARIANT (481)..(481): Replace = Cys
  VARIANT (484)..(484): Replace = Ser
  VARIANT (485)..(485): Replace = Pro or nothing
  VARIANT (486)..(486): Replace = Glu
  VARIANT (488)..(488): Replace = Pro, Asp or Asn
  VARIANT (490)..(490): Replace = Val
  VARIANT (491)..(491): Replace = Glu
  VARIANT (494)..(494): Replace = Ile
  VARIANT (498)..(498): Replace = His
  VARIANT (506)..(506): Replace = Lys
  VARIANT (526)..(526): Replace = Thr
  VARIANT (527)..(527): Replace = Cys or Ala
  VARIANT (530)..(530): Replace = Ser
  VARIANT (533)..(533): Replace = Asn
  VARIANT (534)..(534): Replace = Ala or Val
  VARIANT (535)..(535): Replace = Asn
  VARIANT (539)..(539): Replace = Val
  VARIANT (540)..(540): Replace = Arg
  VARIANT (543)..(543): Replace = Glu
  VARIANT (578)..(578): Replace = Phe
  VARIANT (584)..(584): Replace = Leu
  VARIANT (585)..(585): Replace = Ser
  VARIANT (587)..(587): Replace = Arg or Tyr
  VARIANT (588)..(588): Replace = Gln or nothing
MSTKHLFNETDELVLKSLEGVQASRSASILSHRFKVLYNGSHSADRVAVLSGGGSGHEPAHAGFVGDNMLTGAICGPVFASPSAKQVEAGCKLVPSDKGHILVVTNYTGDMLHFGLAAEKLKSQGHKVGIIKSADDVAVDRKSGGLVGRRGLAGTVLLDKIVGGAAWDKLSFDECMAIGTEVAENTATASIGLDYCHVPGRSVENHVSLAQEECQFGLGIHNEPGVKTMNPVPAPESMVETLLKYLVSQDDSERSFVKFKEGDEVILLANNLGGISTIEMRAAVQLAREQLEKTHKIKSVRVLCGTFMSSLNAPGFSITLVNLSNGSHSKNVLKYLDAVSDAPAWVNTSPLTSSVKPFVSEDKIFDDETSSNIKAPVLDVPEQTIIAALTQASQNIIKAEPQLTAWDTEMGDGDCGHTIEHGCRALLEYLNKNKSDPKALEIIPIVRAVVHITEEDMGGTLGAIFGIFFASFLNALLLDPLSHKTDVSVTDKLVNAANTGLESLMNHTPARPGDRTVMDVLIPYVQSLVATKDIKEAALKAKQAAEGTKKIKPRLGRAVYVGEKDGELPPDPGAWAVYELVDGFANHK

SEQ ID NO: 3 - protein, 588 aa, Yarrowia galli; identical to SEQ ID NO: 2

SEQ ID NO: 4 - protein, 585 aa, Yarrowia yakushimensis
MSTKHLFNETDELVLKSLEGVQASRQASILSHRFKVLYNGSHTSDRVAVLSGGGSGHEPAHAGFVGDNMLTGAICGPVFASPSAKQVEAGCKLVPSDKGHILVVTNYTGDMLHFGLAAEKLKSQGHKVGIIKSADDVAVDRKSGGLVGRRGLAGTVLLDKIVGGAAWDKLSFDECMAIGTEVSDNTATASIGLDYCHVPGRSVENHVSLVQDECQFGLGIHNEPGVKTLNPVPAPETMVRTLLDYLVSQDDHERSFVKFKQGDEVILLANNLGGISTIEMRAAVQLAREQLEKTHKIKPVRVLCGTFMSSLNAPGFSITLVNLSNGSHSNKVLQYLDAVSDAPAWVNVASPVSVKPFVNEDKIFDDDISNLKAPNLDVSTQTVIAALTQASENIIKAEPQLTAWDTEMGDGDCGHTIEHGCRALLEYLHKNQNNTTALEIIPIVRDIVHITEEDMGGTLGAIFGIFFASFLNALLSDPLSHKTDVDVTDKLINAAHTGLESLMKHTPARPGDRTVMDVLIPYVTALVATKDVKEAALRAKQAAEGTKKIKPRLGRAVYVGEKDGELPPDPGAWAVYELVDGFANY

SEQ ID NO: 5 - protein, 585 aa, Yarrowia alimentaria
MSTKHLFNETDELVLKSLEGVQASRSASILSNRFKVLYNGNHTKDRVAVLSGGGSGHEPAHAGFVGDNMLTGAICGPVFASPSAKQVEAGCKLVPSDKGHILVVTNYTGDMLHFGLAAEKLKSQGHKVGIIKSADDVAVDRKSGGLVGRRGLAGTILLDKIVGAAAFAKLSFEECMEIGSEVADNTATASIGLDYCHVPGRSVEKHSSLGQDECQFGLGIHNEPGVKTLSPIPSPDKLVETLLQYIVSQDDAQRSFVKFKEGDEVILLANNLGGISVIEMRAAVQLAVEQLEKTYKIASVRVLCGTYMSSLNAPGFSLTLVNLSNGTHSKQVLEHLDATCDAPAWVNISPPVSVKPFVSEDTIFDESASTLKEPVLDVDQKTLVAALKQASANIVKAEPQLTEWDTEMGDGDCGHTIEHGCKALVSYLDKNKNDPKALEIIPIVRAVVHITEEDMGGTLGAIFGIFFASFLNALLLDPLSHKEVPVTDKLVNAANTGLESLMKHTPARPGDRTVMDVLIPYVQSLVATKDAKEAALKAKQAAEGTKKIKPRLGRAVYVGEKDGELPPDPGAWAVFELVDGFANHQ

SEQ ID NO: 6 - protein, 586 aa, Yarrowia phangnensis
MSTKHLFNETDDLVVKSLKGVQASRSASILSSRFKVLYNGLHTSERVAVLSGGGSGHEPAHAGFVGDNMLTGAICGPVFASPSAKQVEAGCKLVPSDKGHILVVTNYTGDMLHFGLAAEKLKAQGHNVGIIKSADDVAVDKKSGGLVGRRGLAGTVLLDKIVGAAAWDKLSFEECMEIGTYVAENTATASIGLDFCHVPGRSVESHSSLGQNECQFGLGIHNEPGVKTLSPVPNPDTLIETLLSYILSQDDPERSFVKFKENDEVILLANNLGGISTIEMRAAVLLAKEQLENTHNIKAVRVLCGTFMSSLNAPGFSLTLVNLSNGSHQNSVLKYLDAVSDAPAWVNVASPTSVKPFVDEECIFDEDYSQVIAPTIPIDETELVSALTQASENIIKAEPQLTAWDTEMGDGDCGHTIEHGCRALLKYLENNKGNSEALKIIPIVREIVHITEEDMGGTLGAIFGIFFASFLNALLHDHCSHSPDVPVVEKLVNAANTGLESLMKHTPARPGDRTVMDVLIPYVQCLVATKNANEAAVKAKEAAEGTKKIKPRLGRAVYVGEKDGELPPDPGAWAVYELVDGLSNRK

SEQ ID NO: 7 - DNA, 942 bp, Yarrowia lipolytica
atggtttcttcagccgctacttctgctctgcccatctcggcaccctacaccttctaccctcaggctcgagttcctgcccccaagaagctcgttggactcaatgctgctctggaggcccagaagaaccccgagttcgaggtgaagcccgagatctttaaggagttctctctgcccgacggtgttgccattgtcaccggtggaaactccggtattggtcttgagtactcagtctgcctcgccgagctcggtgccactgtctactgtcttgacatgcccgagactccctctgaggagttcctggcttgccagtcctacgttaagcgaatgcccggcaacgcctctctggtcttcaagcgagccgacgtcactgacgaggagactatgaactccctcttccagaacattgccgagacccacggcaagattgacgttgtcatcgctaacgccggtgtgcttggacctcgagcctcttgcaacgagtaccccgctgactggttccgaaaggtcatggacgtcaacgtcaccggtgtctttatcaccgcccaggccgcctctcgacagatgattgccaccaagacttctggttctatcattgtcaccgcctccatgtccggctccattgtcaaccgagacatgccctggtgcgcctacaacgcctccaaggccgctgctgctcatcttgtcaagtccatggctgctgagctcggccagtttgagattcgagtcaactccatctcccccggtcacatccagactgctatgactgacgtctgtcttgacgctgagcccggtcttggtaaccagtgggccttccagaaccccatgggccgacttggaggtgtctccgagcttcgaggagtctgcgcctaccttgcatcttccgcctcctcctacaccaccggctctgacattcttgtctgcggtggccaccacgtctggtaa

SEQ ID NO: 8 - protein, 503 aa, Yarrowia lipolytica
MSSYVGALDQGTTSTRFILFSPDGKPVASHQIEFTQIYPHPGWVEHDPEELVSSCLECMSSVAKEMRTQGIKVADVKAIGITNQRETTVLWDIETGQPLYNAIVWSDARTGDTVKKLEAQPGADEIPKLCGLPLSTYFAGVKVRWILDNVKEARECYDRGKLAFSTIDSWLLYNLTGGLNGGAHITDTSNASRSMFMNIETLKYDEKLIKFFGVEKLILPKIVSSAEVYGRIGTGPFANIPLAGCLGDQSAALVGQKAFEPGQAKNTYGTGCFLLYNAGEKPIISNNGLLTTVGYHFKGQKPVYALEGSISVAGSCIKWLRDNIGLIESSEQIGELASQVDDSAGVVFVTALSGLFAPYWRTDARGTILGLTQFTTKAHICRAALEATCFQTRAILDAMAKDSGKPFTKLRVDGGMTNSDIAMQIQADILGIEVERPAMRETTALGAAIAAGFAVGVWKSIEDLKDINTEGMTEFASKTNEEERAAMMKQWNRGIERAVGWLE

SEQ ID NO: 9 - protein, 612 aa, Yarrowia lipolytica
MFRTIRKPAWAAAAAVAAAGAGAVALSVPAQAQEELHKKHKFTVPPVAAEPPSRAAQLEKMKTEEFDLVVVGGGATGSGIALDAVTRGLKVALVERDDFSCGTSSRSTKLIHGGVRYLEKAVWNLDYNQYELVKEALHERKVFLDIAPHLTFALPIMIPVYTWWQLPYFWMGVKCYDLLAGRQNLESSYMLSRSRALDAFPMLSDDKLKGAIVYYDGSQNDSRMNVSLIMTAVEKGATILNHCEVTELTKGANGQLNGVVAKDTDGNAGSFNIKAKCVVNATGPFTDSLRQMDDKNTKEICAPSSGVHIILPGYYSPKKMGLLDPATSDGRVIFFLPWQGNTLAGTTDQPTKITANPIPSEEDIDFILNEVRHYVEGKVDVRREDVLAAWSGIRPLVRDPHAKNTESLVRNHLITYSESGLVTIAGGKWTTYRQMAEETVDACIAKFGLKPEISAKAVTRDVKLIGAKDWTPLTYIDLIQQEDLDPEVAKHLSENYGSRAFTVASLAEMPTPEPGVIPQSTLTKGKRILYPYPYLDAECKYSMKYEYATTAIDFLARRTRLAFLNAAAAYEALPEVIEIMAKELQWDEARKEQEFNTGVEYLYSMGLTPKDK

SEQ ID NO: 10 - protein, 247 aa, Yarrowia lipolytica
MSRTFFVGGNFKMNGSLESIKAIVERLNASELDPKTEVVISPPFPYLLLAKESLKKPTVSVAGQNSFDKGDGAFTGEVSVAQLKDVGAKWVILGHSERRTINKESSEWIADKTKYALDNGLDVILCIGETIDEKKAGKTLDVVRSQLDPVIAKIKDWSNVVIAYEPVWAIGTGLAATAEDAQQIHHEIRAYLKDKIGAQADKVRIIYGGSVNGKNSGTFKDKSDVDGFLVGGASLKPEFVDIINSRL

SEQ ID NO: 11 - protein, 694 aa, Yarrowia lipolytica
MAPQFSKTDETAINTIRTLAIDAVAKANSGHPGAPMGLAPVAHVLWNYYMNFTSSNPEWINRDRFILSNGHACMLHYSLLHLFGYDITIDDLKNFRQLNSKTPGHPEAETPGIEVTTGPLGQGVSNAVGFAIAQAHLGATYNKPGYDIINNYTYCIFGDGCMMEGVASEAMSLAGHLQLGNLITFYDDNHISIDGDTNVAFTEDVSQRLEAYGWEVIWVKDGNNDLAGMAAAIEQAKKSKDKPTCIRLTTIIGYGSLQQGTHGVHGSPLKPDDIKQFKEKVGFNPEETFAVPKETTDLYAKTIDRGANAEKEWNELFAKYGKEYPKEHSEIIRRFKRELPEGWEKALPTYTPADNAVASRKLSEIVLTKIHEVLPELVGGSADLTGSNLTRWKDAVDFQPPVTHLGDYSGRYIRYGVREHGMGAIMNGMNAYGGIIPYGGTFLNFVSYAAGAVRLSALSGHHVIWVATHDSIGLGEDGPTHQPIETVAWLRATPNLSVWRPADGNETSAAYYKAITNYHTPSVLSLTRQNLPQLEGSSIEKASKGGYQLISEDKGDIYLVSTGSEVAICVAAAKLLKEKKGITAGVISLPDWFTFEQQSLEYRKSVFPDGIPMLSVEVYSDFGWSRYSHQQFGLDRFGASAPFQQVYDAFEFNAEGVAKRAEATINYYKGQTVKSPIQRAFDPIDVNTRPGHGV

SEQ ID NO: 12 - protein, 282 aa, Candida magnoliae
MSSTYTLTRLSAPSMVLNSGSQIPAVGYGLWKQQGSEAKDSVRCAIESGYRHLDCATAYQNHKEVGQAIREAGVPRDELWITSKVWGTHFDNPEEGLDDILEELGVEYLDLLLLHLPVAFKRNPEDPKQLRGLPVDHDMKYADVWARMEKLPKSKVRNIGVSNLTVRALDELLQTAKVTPAVNQVEMHPNLPQKKLLDYCKSKGIVVQAYSPLAQGQHENPVVTDIADDLGVSPAQVVLSWGALRGTNILPKSSTPSRIRENLELIQLSDDHMRRIDALARR

SEQ ID NO: 13 - DNA, 29 bp, Artificial
  Primer GSP1-L
tctcggtggtcaatgcgtcagaagatatc

SEQ ID NO: 14 - DNA, 28 bp, Artificial
  Primer GSP2-L
agccgagtgaatgttgcctgccgttagt

SEQ ID NO: 15 - DNA, 27 bp, Artificial
  Primer GSP1-R
agcgttcgccaattgctgcgccatcgt

SEQ ID NO: 16 - DNA, 29 bp, Artificial
  Primer GSP2-R
acactaccgaggttactagagttgggaaa

SEQ ID NO: 17 - DNA, 22 bp, Artificial
  Primer AP1
gtaatacgactcactatagggc

SEQ ID NO: 18 - DNA, 19 bp, Artificial
  Primer AP2
actatagggcacgcgtggt

SEQ ID NO: 19 - DNA, 22 bp, Artificial
  Primer DISR1
tgtagcacctgggtcaacattt

SEQ ID NO: 20 - DNA, 21 bp, Artificial
  Primer DISR2
tccgatgacctgactagtgcg

SEQ ID NO: 21 - DNA, 22 bp, Artificial
  Primer CHK1
gattgctccgtttgtaagtaca

SEQ ID NO: 22 - DNA, 20 bp, Artificial
  Primer ZETA1
tggtcctgttccacctgaac

SEQ ID NO: 23 - DNA, 33 bp, Artificial
  Primer GUT1 F1
gacggatccatgtcttcctacgtaggagctctc

SEQ ID NO: 24 - DNA, 21 bp, Artificial
  Primer GUT1 R1
gttatccagaatccatcggac

SEQ ID NO: 25 - DNA, 20 bp, Artificial
  Primer GUT1 F2
ggtccgatggattctggata

SEQ ID NO: 26 - DNA, 29 bp, Artificial
  Primer GUT1 R2
gaccctaggttactcaagccagccaacag

SEQ ID NO: 27 - DNA, 30 bp, Artificial
  Primer TKL1 F1
cgaggatccatggctccccaattttcaaag

SEQ ID NO: 28 - DNA, 34 bp, Artificial
  Primer TKL1 R1
gccacagcatcaatgccaaggttcggatggtgtt

SEQ ID NO: 29 - DNA, 44 bp, Artificial
  Primer TKL1 F2
atcaacaccatccgaaccttggctattgatgctgtggccaaggc

SEQ ID NO: 30 - DNA, 32 bp, Artificial
  Primer TKL1 R2
gttcttgagatcatcaatagtgatgtcgtagc

SEQ ID NO: 31 - DNA, 32 bp, Artificial
  Primer TKL1 F3
gctacgacatcactattgatgatctcaagaac

SEQ ID NO: 32 - DNA, 29 bp, Artificial
  Primer TKL1 R3
gaccctaggttagacaccgtggccgggtc

SEQ ID NO: 33 - DNA, 22 bp, Artificial
  Primer URA3 F
aggaagaaaccgtgcttaagag

SEQ ID NO: 34 - DNA, 22 bp, Artificial
  Primer LEU2 F
taagtcgtttctacgacgcatt

SEQ ID NO: 35 - DNA, 23 bp, Artificial
  Primer 61 Stop
gtagatagttgaggtagaagttg

SEQ ID NO: 36 - DNA, 23 bp, Artificial
  Primer EYK-PF
gttgtgtgatgagaccttggtgc

SEQ ID NO: 37 - DNA, 37 bp, Artificial
  Primer EYK-PR
aaaggccatttaggccgcagctcctccgacaatcttg

SEQ ID NO: 38 - DNA, 37 bp, Artificial
  Primer EYK-TF
taaggccttgatggccacaagtagagggaggagaagc

SEQ ID NO: 39 - DNA, 22 bp, Artificial
  Primer EYK-TR
gtttaggtgcctgaagacggtg

SEQ ID NO: 40 - DNA, 39 bp, Artificial
  Primer LPR-F
ataggcctaaatggcctgcatcgatctagggataacagg

SEQ ID NO: 41 - DNA, 42 bp, Artificial
  Primer LPR-R
ataggccatcaaggccgctagatagagtcgagaattaccctg

SEQ ID NO: 42 - DNA, 20 bp, Artificial
  Primer GUT1-L-q
ccctgtccacctactttgcc

SEQ ID NO: 43 - DNA, 20 bp, Artificial
  Primer GUT1-R-q
ttggaggtgtcggtgatgtg

SEQ ID NO: 44 - DNA, 20 bp, Artificial
  Primer TKL1-P-L-q
cagcaacacagatggcaacc

SEQ ID NO: 45 - DNA, 22 bp, Artificial
  Primer TKL1-T-R-q
cgagacctccgctgcttactac

SEQ ID NO: 46 - DNA, 21 bp, Artificial
  Primer ACT-F
ggccagccatatcgagtcgca

SEQ ID NO: 47 - DNA, 18 bp, Artificial
  Primer ACT-R
tccaggccgtcctctccc

SEQ ID NO: 48 - DNA, 945 bp, Lipomyces starkeyi
atgcctgctgtcgacggaacaactggcaattcagcaaaactcagcacttcggctgtacgattcccagtcaaactcgtgcctgtcccggtcaaagacgtcggtgtcaatgcagctctcaaagcccagtcggatccgtcattcgaagtcaagccacgtattttcgaggagtttgcacttactggacaggtcgcaatcgtcacgggcgggaacggcggtcttggcctcgagtttgcgatcgtcctcgcggagctaggcgcgaaggtctacgccatcgatctgcccgctactccgtcctccgactttgtggccgcggtcaagtatgtcaagcgacttggttcgtccctccagtatcggccgtccgacgtgagtaagcaagaaatcatcagcgcaaccatcggcgagattgctgccgagaacgatggcaagatacatgtttgtgtggcggcagcaggaattttaggaattgaggccgactgtaccgattatcccgccaatatgttcgagaaggttatggatgttaactgcaacggcgtgtttttcacagctcaggcagcggctaagcagatgaaacaacaggacatcgcaggcagcattattttgattgcgagcatgtcgggcagtgtcaccaaccgagatatgaattggattccgtacaatgcttccaaatcagcagtgatccagattgcacgctcgatggcctgcgaacttgggccagcgggtattcgcgtcaactcgctctcgccgggccatatccgcacgaagatgaccgctgccgtactggacacccaaccggaaatggaagaattttgggcgagcttgaatccgttgggccgtattggtgccgtacatgagttgagaggtgtcatcgcgtggttggcgagcgaggcgtcgacgttctgcactggtagcgatatccttgtaactggcgggcatacgatctggtag

SEQ ID NO: 49 - DNA, 945 bp, Lipomyces starkeyi
atgcctgccgtcaacggaacaagtggcaattcagcaaaactcagcacttctgctgtacgatccccagtcaaacttgtgcctgtcccggtcaaagacgtcggtgtcaatgcagcgctcaaagcacagtcggatccgtcattcgaagtcaagccgcatattttcgaagagtttgcacttaccggacaggtcgcaatcgtcactggcgggaacggcggtcttggcctcgagtttgcgatcgtcctcgcagagcaaggcgccaaggtctacgccatcgatctgcccgctacgccgtcttctgactttgtggccgcagtcaattatgtcaagcgacttggttcgtccctccagtatcggtcgtccaacgtgagtaagcaggaaatggtcaacgcgaccatctgcgagattgctgccgagaacgatggcaagatacatgtttgtgtggcagcagcaggaattttgggaattgaggccgaatgtaccgattatcccgccaatatgttcgagaaggttatggatgttaactgcaacggtgtgtttttcacagctcaggcagcggctaagcagatgaaacaacaggacatcgcaggcagcattattttgattgcgagcatgtcgggaagtgtcaccaaccgagaaatgaattggagtccgtacaatgcttccaaatcagcagtgatccagattgcacgctcgatggcctgcgaacttgggcaagcgggtattcgcgtcaactcgctctcgccgggccatatccgcacgaaaatgaccgctgccgtactggacattcaaccggaaatggaagaattttgggcgagcttgaatcctttgggccgtattggtgccgtacatgagttgagaggtgtcattgcgtggttggcgagcgacgcgtcgacgttctgcactggtagcgatatccttgtgactggcgggcatacgatatggtaa

SEQ ID NO: 50 - DNA, 35 bp, Artificial
  Primer EYD_Surexp_F
gacggatcccacaatggtttcttcagccgctactt

SEQ ID NO: 51 - DNA, 29 bp, Artificial
  Primer EYD_surexp_R
gaccctaggttaccagacgtggtggccac

(19) United States
(12) Patent Application Publication
(10) Pub. No.: US 2019/0136278 A1
(43) Pub. Date: May 9, 2019

(54) MUTANT YEAST STRAINS WITH ENHANCED PRODUCTION OF ERYTHRITOL OR ERYTHRULOSE

(71) Applicants: INSTITUT NATIONAL DE LA RECHERCHE AGRONOMIQUE, Paris (FR); UNIVERSITE LIBRE DE BRUXELLES, Bruxelles (BE); UNIVERSITE DE LIEGE, Liege (BE)

(72) Inventors: Jean-Marc NICAUD, Trappes (FR); Patrick FICKERS, Liege (BE); Frederic CARLY, Bruxelles (BE)

(21) Appl. No.: 16/099,960

(22) PCT Filed: May 5, 2017
(86) PCT No.: PCT/EP2017/060823

§ 371 Date: Nov. 8, 2018

(30) Foreign Application Priority Data
May 10, 2016 (EP) 16305539.5

(51) Int. Cl. C12P 19/02; C12N 9/04; C12N 9/12; C12N 9/90; C12N 9/10; C12N 9/16; C12P 7/18

(52) U.S. Cl. CPC C12P 19/02; C12N 9/0006; C12N 9/1205; C12P 7/18; C12N 9/1022; C12N 9/16; C12N 9/90

(57) ABSTRACT

The invention relates to a method for enhancing the erythritol and/or erythrulose productivity and/or yield of an erythritol and/or erythrulose-producing yeast strain, such as Yarrowia lipolytica, comprising inhibiting in said yeast strain the expression or the activity of an endogenous L-erythrulose kinase and/or erythritol dehydrogenase. The invention also relates to a mutant yeast strain obtained by said method.

The present invention relates to mutant yeast strains, in particular mutant Yarrowia strains, having an enhanced erythritol and/or erythrulose production and/or yield. The present invention also relates to means and methods for obtaining these mutant yeast strains.

Erythritol is a four-carbon polyol naturally found in fruits, seaweeds or mushrooms, and produced by many osmophilic microorganisms as a protection against osmotic stress. In the food industry, erythritol is used as a food additive because of its sweetening properties. It is 60-70% as sweet as sucrose but it has low energy value, it is non-cariogenic and it does not affect glycemia. A large number of toxicological and clinical studies have shown its safety for human consumption, with no negative effect observed on health. It would also have antixodiant properties.

Industrially, erythritol is mainly produced by fermentation using osmophilic yeasts grown under high osmotic pressure. Most processes use glucose as a carbon source and are conducted either in batch or fed-batch fermentation mode (Moon et al., 2010). Erythritol producer include Aurobasidium sp. (Ishizuka et al., 1989), Trigonopsis variabilis (Kim et al., 1997), Torula sp. (Lee et al., 2000), Candida magnoliae (Ryu et al., 2000) or Pseudozyma tsubakaensis (Jeya et al., 2009) or Yarrowia (patent application EP 0 845 538).

Erythrulose (S-1,3,4-thihydroxy-2-butanone, L-glycero-2-tetrulose) is used in some self-tanning cosmetics, mostly in combination with dihydroxyacetone. Erythrulose reacts with amino acids from proteins of the stratum corneum and epidermis in a process similar to Maillard reaction. Erythrulose can also be used as a multifunctional chiron for the synthesis of polyoxygenated molecules such as macrolide and polyethers antibiotics.

Erythrulose can be obtained by chemical synthesis from formaldehyde and dihydroxyacetone by phosphate catalysis in neutral aqueous medium. It can also be synthesized using a transketolase catalysed reaction of lithium hydroxypyruvate and glycolaldehyde to erythrulose. A bioprocess of erythrulose synthesis from erythritol in the bacteria Gluconobacter frateurii was reported in the literature (Moonmangnee et al., 2002; Mizanur et al., 2001).

Yarrowia lipolytica is a non-conventional dimorphic yeast, belonging to the subphylum Saccharomycotina. Y. lipolytica is well-known for its ability to use n-alkanes and fatty acids as carbon source, namely glucose, fructose and mannose (Barth and Gaillardin 1997; Nicaud 2012). Thanks to its ability to secrete high amounts of proteins and metabolites of interest, Y. lipolytica has been used in several industrial applications, including heterologuous protein production and citric acid production (Fickers et al., 2005; Zinjarde, 2014). Y. lipolytica gave good results for erythritol production, and has the advantage of using raw glycerol as a carbon source instead of glucose (Rymowicz et al., 2008). Raw glycerol, a byproduct of biodiesel production, is a renewable carbon source that it is both cheaper and more efficient than glucose for erythritol production (Tomaszewska et al., 2012, Rywhiska et al., 2013).

Recently, Yarrowia lipolytica, in particular the acetate-negative mutant Y. lipolytica Wratislavia K1 (isolated from continuous citric acid fermentation with the parent strain of Y. lipolytica Wratislavia 1.31 in chemostat experiments) has been reported for erythritol production in fed-batch cultivations by using glycerol as the carbon source (Rymowicz et al., 2008; Tomaszewska et al., 2012). Carly et al. (2015) disclosed a genetically modified Y. lipolytica overexpressing glycerol kinase gene (GUT1) that showed a higher erythritol productivity.

The inventors have identified an essential gene of the erythritol catabolism in Y. lipolytica, YALI0F01606g, which encodes the protein referred to as SEQ ID NO: 1. They demonstrated that the loss of this gene is sufficient to remove the ability of Y. lipolytica to grow on erythritol. Although annotated as a dihydroxyacetone kinase, the properties of this gene indicate that it might code for an L-erythrulose kinase (EYK), an enzyme of the erythritol catabolism pathway, responsible for the conversion of L-erythrulose into L-erythrulose phosphate. To the knowledge of the inventors, it is the first EYK sequence (i.e. YALI0F01606g, gene EYK1) known in a living organism. Regardless of this, the results clearly showed that disrupting the YALI0F01606g gene have a positive effect on erythritol productions. A Y. lipolytica strain disrupted in the YALI0F01606g gene (FCY001 strain) displayed a higher yield of at least 25%, in particular from 25% to 35%, and a higher specific productivity of about 30% than the wild-type strain W29. Even more, unlike the wild-type strain, erythritol concentration remained stable in the medium over time, making it a well-suited strain for industrial production without erythritol re-consumption. Further, said FCY001 strain is able to produce erythrulose in high biomass and high erythritol concentration conditions.

Accordingly, the present invention provides a method for enhancing the erythritol or erythrulose productivity and/or yield (advantageously the erythritol or erythrulose productivity and yield) of an erythritol and/or erythrulose-producing yeast strain, wherein said method comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1).

L-erythrulose kinase (EC 2.7.1.27) belongs to the family of transferases, specifically those transferring phosphorus-containing groups (phosphotransferases) with an alcohol group as acceptor. The systematic name of this enzyme class is ATP:erythritol 4-phosphotransferase. This enzyme is also called erythritol kinase (phosphorylating). It catalyses the following reaction which requires ATP:

ATP+erythritol [symbol] ADP+D-erythritol 4-phosphate

Methods for determining whether an enzyme has an L-erythrulose kinase (EC 2.7.1.27) activity are known in the art. By way of example, one can use the method described in Wu (2011).

In all the aspects of the present invention, the L-erythrulose kinase (EC 2.7.1.27) is preferably of sequence SEQ ID NO: 1.

In a preferred embodiment, the L-erythrulose kinase comprises or consists of the consensus amino acid sequence SEQ ID NO: 2. This sequence SEQ ID NO: 2 corresponds to the consensus amino acid sequence obtained by aligning the L-erythrulose kinase from the strains Yarrowia lipolytica CLIB122 (YALI_EYK1 of SEQ ID NO: 1), Yarrowia galli CBS 9722 (YAGA_EYK1 of SEQ ID NO: 3), Yarrowia yakushimensis CBS 10253 (YAYA_EYK1 of SEQ ID NO: 4), Yarrowia alimentaria CBS 10151 (YAAL EYK1 of SEQ ID NO: 5) and Yarrowia phangnensis CBS 10407 (YAPH_EYK1 of SEQ ID NO: 6).

The L-erythrulose kinase of SEQ ID NO: 3 (YAGA_EYK1), SEQ ID NO: 4 (YAYA_EYK1), SEQ ID NO: 5 (YAAL EYK1) and SEQ ID NO: 6 (YAPH_EYK1) have respectively 96.77%, 91.62%, 87.22% and 85.01% identity with the polypeptide of sequence SEQ ID NO: 1 (YALI_EYK1).

Unless otherwise specified, the percent of identity between two protein sequences which are mentioned herein is calculated from the BLAST results performed either at the NCBI (http://blast.ncbi.nlm.nih.gov/Blast.cgi) or at the GRYC (http://gryc.inra.fr/) websites using the BlastP program with the default BLOSUM62 parameters as described in Altschul et al. (1997).

Advantageously, if the yeast strain is a Yarrowia strain, the L-erythrulose kinase is selected from the group consisting of SEQ ID NO: 1, SEQ ID NO: 3, SEQ ID NO: 4, SEQ ID NO: 5 and SEQ ID NO: 6.

The L-erythrulose kinase from the strain Y. lipolytica CLIB122 (YALI_EYK1) of SEQ ID NO: 1 is encoded in Y. lipolytica by the gene YALI0F01606g.

The erythritol and/or erythrulose-producing yeast strain (i.e., a yeast strain capable of producing erythritol and/or erythrulose) include osmophilic yeast strains, which are capable of growing in media with high osmotic pressure, i.e., in the presence of high sugar or salts concentration (see Moon et al., 2010). They generally belong to the genus selected from the group consisting of Aurobasidium, Candida, Moniliella (or Trichosporonoides), Pseudozyma, Torula, Trichosporon, Trigonopsis or Yarrowia. More specifically, examples include Aureobasidium sp., Candida magnolia, Moniliella sp., Moniliella tomentosa var. pollinis, Pseudozyma tsubakaensis, Torula sp, Trichosporon sp., Trigonopsis variabilis, Yarrowia sp., Yarrowia alimentaria Yarrowia galli, Yarrowia lipolytica, Yarrowia phangnensis and Yarrowia yakushimensis. In a preferred embodiment, the erythritol and/or erythrulose-producing yeast strain is a Yarrowia strain, more preferably is selected from the group consisting of Y. lipolytica, Y. galli, Y. yakushimensis, Y. alimentaria and Y. phangnensis, most preferably is a Y. lipolytica strain.

Said Yarrowia strain can be auxotrophic for leucine (Leu−) and optionally for the decarboxylase orotidine-5′-phosphate (Ura−).

Advantageously, the erythritol and/or erythrulose-producing yeast strain is selected from the group consisting of Y. lipolytica, Y. galli, Y. yakushimensis, Y. alimentaria and Y. phangnensis and the L-erythrulose kinase is respectively selected from the group consisting of SEQ ID NO: 1, 3, 4, 5 and 6.

The method for enhancing the erythritol or erythrulose productivity and/or yield of an erythritol and/or erythrulose-producing yeast strain according to the present invention can further comprises overexpressing in said strain at least one gene encoding enzyme involved in the pathway of erythritol biosynthesis and/or at least one gene encoding enzyme involved in the pathway of erythrulose biosynthesis and/or inhibiting the expression or activity of at least one endogenous gene involved in erythritol catabolism.

Enzymes involved in the pathway of erythritol biosynthesis are described in Moon et al., 2010. Advantageously, said enzyme involved in the pathway of erythritol biosynthesis is selected from the group consisting of:
a glycerol kinase (EC 2.7.1.30), advantageously a yeast glycerol kinase, more advantageously an endogenous glycerol kinase of said strain,
a glycerol-3P dehydrogenase (EC 1.1.5.3), advantageously a yeast glycerol-3P dehydrogenase, more advantageously an endogenous glycerol-3P dehydrogenase of said strain,
a triose isomerase (EC 5.3.1.1), advantageously a yeast triose isomerase, more advantageously an endogenous triose isomerase of said strain,
a transketolase (EC 2.2.1.1), advantageously a yeast transketolase, more advantageously an endogenous transketolase of said strain,
an erythrose 4 phosphate phosphatase (EC 3.1.3.23), such as an erythrose 4 phosphate phosphatase corresponding to the enzyme named erythrose-4-phosphatase in Kuznetsova et al. (2006) or erythrose-4-phosphate phosphatase in Moon et al. (2010), advantageously an endogenous erythrose 4 phosphate phosphatase of said strain,
an erythrose reductase (EC 1.1.1.21), advantageously a yeast erythrose reductase, more advantageously an endogenous erythrose reductase of said strain, and
an invertase (EC 3.2.1.26), advantageously a yeast invertase, more advantageously the S cerevisiae invertase.

More advantageously, said enzyme involved in the pathway of erythritol biosynthesis is a glycerol kinase as defined above and/or a transketolase as defined above, and even more advantageously the enzymes involved in the pathway of erythritol biosynthesis are a glycerol kinase as defined above and a transketolase as defined above.

Advantageously, said enzyme involved in the pathway of erythrulose biosynthesis is selected from the group consisting of:
a glycerol kinase (EC 2.7.1.30), advantageously a yeast glycerol kinase, more advantageously an endogenous glycerol kinase of said strain,
a glycerol-3P dehydrogenase (EC 1.1.5.3), advantageously a yeast glycerol-3P dehydrogenase, more advantageously an endogenous glycerol-3P dehydrogenase of said strain,
a triose isomerase (EC 5.3.1.1), advantageously a yeast triose isomerase, more advantageously an endogenous triose isomerase of said strain,
a transketolase (EC 2.2.1.1), advantageously a yeast transketolase, more advantageously an endogenous transketolase of said strain,
an erythrose 4 phosphate phosphatase (EC 3.1.3.23), such as an erythrose 4 phosphate phosphatase corresponding to the enzyme named erythrose-4-phosphatase in Kuznetsova et al. (2006) or erythrose-4-phosphate phosphatase in Moon et al. (2010), advantageously an endogenous erythrose 4 phosphate phosphatase of said strain,
an erythrose reductase (EC 1.1.1.21), advantageously a yeast erythrose reductase, more advantageously an endogenous erythrose reductase of said strain,
an invertase (EC 3.2.1.26), advantageously a yeast invertase, more advantageously the S cerevisiae invertase, and
an erythritol dehydrogenase (EC 1.1.1.9), such as an erythritol dehydrogenase described in Paradowska and Nitka (2009), advantageously a yeast erythritol:NAD+2-oxydoreductase or more precisely a yeast erythritol dehydrogenase, more advantageously an endogenous erythritol:NAD+2-oxydoreductase of said strain or more precisely a yeast erythritol dehydrogenase of said strain.

More advantageously, said enzyme involved in the pathway of erythrulose biosynthesis is an erythritol dehydrogenase as defined above, and even more advantageously the enzymes involved in the pathway of erythrulose biosynthesis are an erythritol dehydrogenase as defined above and a glycerol kinase as defined above and/or a transketolase as defined above, and even more advantageously the enzymes involved in the pathway of erythrulose biosynthesis are an erythritol dehydrogenase as defined above and a glycerol kinase as defined above and a transketolase as defined above.

Advantageously, said enzyme involved in the pathway of erythritol catabolism, in particular in bioconversion of erythritol into erythrulose, is an erythritol dehydrogenase (EC 1.1.1.9), such as an erythritol dehydrogenase described in Paradowska and Nitka (2009), advantageously a yeast erythritol:NAD+2-oxydoreductase or more precisely a yeast erythritol dehydrogenase, more advantageously an endogenous erythritol:NAD+2-oxydoreductase of said strain or more precisely a yeast erythritol dehydrogenase of said strain.

Erythritol dehydrogenase (EC 1.1.1.9) belongs to the family of oxidoreductase, specifically to polyol deshydrogenase, more specifically erythritol deshydrogenase. The systematic name of this enzyme class is erythritol:NAD+2-oxydoreductase. It catalyses the oxidation of erythritol into erythulose following reaction: erythritol+NAD [symbol] erythrulose+NADH+H.

Methods for determining whether an enzyme has an activity of erythritol dehydrogenase (EC 1.1.1.9) are known in the art. By way of example, one can use the method described in Paradowska and Nitka (2009).

In all the aspects of the present invention, the erythritol dehydrogenase (EC 1.1.1.9) is preferably of sequence SEQ ID NO: 7.

The inhibition of the expression or activity of the endogenous L-erythrulose kinase or of the endogenous erythritol dehydrogenase can be total or partial. It may be obtained in various ways by methods known in themselves to those skilled in the art. The term inhibiting the expression or activity of an endogenous L-erythrulose kinase or of an erythritol dehydrogenase in a yeast strain refers to decreasing the quantity of said enzyme produced in a yeast strain compared to a reference (control) yeast strain wherein the expression or activity of said endogenous L-erythrulose kinase or of said endogenous erythritol dehydrogenase is not inhibited and from which the mutant strain derives.

This inhibition may be obtained by mutagenesis of the endogenous gene encoding said L-erythrulose kinase (EYK1 gene) or said erythritol dehydrogenase (EYD1 gene) using recombinant DNA technology or random mutagenesis. This may be obtained by various techniques, performed at the level of DNA, mRNA or protein, to inhibit the expression or the activity of the L-erythrulose kinase or of the erythritol dehydrogenase.

At the level of DNA, mRNA, this inhibition may be accomplished by deletion, insertion and/or substitution of one or more nucleotides, site-specific mutagenesis, random mutagenesis, targeting induced local lesions in genomes (TILLING), knock-out techniques, or gene silencing using, e.g., RNA interference, antisense, aptamers, and the like.

This inhibition may also be obtained by insertion of a foreign sequence in the EYK1 gene or EYD1 gene, e.g., through transposon mutagenesis using mobile genetic elements called transposons, which may be of natural or artificial origin.

The mutagenesis of the endogenous gene encoding said L-erythrulose kinase (EYK1 gene) or of the endogenous erythritol dehydrogenase can be performed at the level of the coding sequence or of the sequences for regulating the expression of this gene, in particular at the level of the promoter, resulting in an inhibition of transcription or of translation of said L-erythrulose kinase or said erythritol dehydrogenase.

The mutagenesis of the endogenous EYK1 gene or of the endogenous EYD1 gene can be carried out by genetic engineering. It is, for example, possible to delete all or part of said gene and/or to insert an exogenous sequence. Methods for deleting or inserting a given genetic sequence in yeast, in particular in Y. lipolytica, are well known to those skilled in the art (for review, see Barth and Gaillardin, 1996; Madzak et al., 2004). By way of example, one can use the method referred to as POP IN/POP OUT which has been used in yeasts, in particular in Y. lipolytica, for deleting the LEU2 and XPR2 genes (Barth and Gaillardin, 1996). One can also use the SEP method (Maftahi et al., 1996) which has been adapted in Y. lipolytica for deleting the PDX genes (Wang et al., 1999). One can also use the SEP/Cre method developed by Fickers et al. (2003) and described in International application WO 2006/064131. In addition, methods for inhibiting the expression or the activity of an enzyme in yeasts are described in International application WO 2012/001144.

An advantageous method according to the present invention consists in replacing the coding sequence of the endogenous EYK1 gene or of the endogenous EYD1 gene with an expression cassette containing the sequence of a gene encoding a selectable marker. It is also possible to introduce one or more point mutations into the endogenous EYK1 gene or into the endogenous EYD1 gene, resulting in a shift in the reading frame, and/or to introduce a stop codon into the sequence and/or to inhibit the transcription or the translation of the endogenous EYK1 gene or of the endogenous EYD1 gene.

Another advantageous method according to the present invention consists in genetically transforming said yeast strain with a disruption cassette of said endogenous EYK1 gene or of said endogenous EYD1 gene. A suitable disruption cassette for disrupting the endogenous EYK1 gene or the endogenous EYD1 gene contains specific sequences for homologous recombination and site-directed insertion, and a selection marker.

The mutagenesis of the endogenous EYK1 gene or of the endogenous EYD1 gene can also be carried out using physical agents (for example radiation) or chemical agents. This mutagenesis also makes it possible to introduce one or more point mutations into the EYK1 gene or into the EYD1 gene.

The mutated EYK1 gene or the mutated EYD1 gene can be identified for example by PCR using primers specific for said gene.

It is possible to use any selection method known to those skilled in the art which is compatible with the marker gene (or genes) used. The selectable markers which enable the complementation of an auxotrophy, also commonly referred to as auxotrophic markers, are well known to those skilled in the art in the field of yeast transformation. The URA3 selectable marker is well known to those skilled in the art. More specifically, a yeast strain in which the URA3 gene (sequence available in the Génolevures database (http://genolevures.org/) under the name YALI0E26741g or the UniProt database under accession number Q12724), encoding orotidine-5′-phosphate decarboxylase, is inactivated (for example by deletion), will not be capable of growing on a medium not supplemented with uracil. The integration of the URA3 selectable marker into this yeast strain will then make it possible to restore the growth of this strain on a uracil-free medium. The LEU2 selectable marker described in particular in patent U.S. Pat. No. 4,937,189 is also well known to those skilled in the art. More specifically, a yeast strain in which the LEU2 gene (e.g., YALI0000407g in Y. lipolytica), encoding β-isopropylmalate dehydrogenase, is inactivated (for example by deletion), will not be capable of growing on a medium not supplemented with leucine. As previously, the integration of the LEU2 selectable marker into this yeast strain will then make it possible to restore the growth of this strain on a medium not supplemented with leucine. The ADE2 selectable marker is also well known to those skilled in the art. A yeast strain in which the ADE2 gene (e.g., YALI0B23188g in Y. lipolytica), encoding phosphoribosylaminoimidazole carboxylase, is inactivated (for example by deletion), will not be capable of growing on a medium not supplemented with adenine. Here again, the integration of the ADE2 selectable marker into this yeast strain will then make it possible to restore the growth of this strain on a medium not supplemented with adenine. Leu⁻ Ura⁻ auxotrophic Y. lipolytica strains have been described by Barth and Gaillardin, 1996.

In a preferred embodiment, the method for enhancing the erythritol productivity and/or yield of an erythritol-producing yeast strain comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing at least 1, 2, 3, 4, 5, 6 or the 7 enzymes selected from the group consisting of a glycerol kinase, a glycerol-3P dehydrogenase, a triose isomerase, a transketolase, an erythrose 4 phosphate phosphatase, an erythrose reductase and an invertase, preferably overexpressing at least a glycerol kinase or a transketolase. More preferably, the method for enhancing the erythritol productivity and/or yield of an erythritol-producing yeast strain comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing a glycerol kinase and a transketolase.

In another preferred embodiment, the method for enhancing the erythrulose productivity and/or yield of an erythrulose-producing yeast strain comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing at least an erythritol dehydrogenase and optionally at least 1, 2, 3, 4, 5, 6 or the 7 enzymes selected from the group consisting of a glycerol kinase, a glycerol-3P dehydrogenase, a triose isomerase, a transketolase, a fumarase, an erythrose 4 phosphate phosphatase, an erythrose reductase and an invertase. Preferably the method for enhancing the erythrulose productivity and/or yield of an erythrulose-producing yeast strain comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing an erythritol dehydrogenase and a glycerol kinase or a transketolase. More preferably, the method for enhancing the erythrulose productivity and/or yield of an erythrulose-producing yeast strain comprises inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing an erythritol dehydrogenase, a glycerol kinase and a transketolase.

Preferably, said erythritol dehydrogenase is a polypeptide of sequence SEQ ID NO: 7 (YALI_EYD1) or an erythritol dehydrogenase having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 7 (YALI_EYD1).

In another aspect, the present invention is related to a method for enhancing the erythritol productivity and/or yield of an erythritol-producing yeast strain without production of erythrulose, said method comprising inhibiting in said yeast strain the expression or the activity of an endogenous L-erythrulose kinase (EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and inhibiting in said yeast strain the expression or the activity of an endogenous erythritol dehydrogenase (EC 1.1.1.9). Optionally said method comprises overexpressing at least 1, 2, 3, 4, 5, 6 or the 7 enzymes selected from the group consisting of a glycerol kinase, a glycerol-3P dehydrogenase, a triose isomerase, a transketolase, an erythrose 4 phosphate phosphatase, an erythrose reductase and an invertase, preferably overexpressing at least a glycerol kinase or a transketolase, preferably overexpressing a glycerol kinase and a transketolase. Preferably, said endogenous erythritol dehydrogenase is a polypeptide of sequence SEQ ID NO: 7 (YALI_EYD1) or an erythritol dehydrogenase having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 7 (YALI_EYD1).

In another aspect, the present invention is related to a method for enhancing the erythritol productivity and/or yield of an erythritol-producing yeast strain without production of erythrulose, said method comprising inhibiting in said yeast strain the expression or the activity of an endogenous erythritol dehydrogenase (EC 1.1.1.9) and optionally overexpressing in said strain at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21) and an invertase (EC 3.2.1.26), preferably a glycerol kinase and/or a transketolase, even more preferably a glycerol kinase and a transketolase. Preferably, said endogenous erythritol dehydrogenase is a polypeptide of sequence SEQ ID NO: 7 (YALI_EYD1) or an erythritol dehydrogenase having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 7 (YALI_EYD1).

Advantageously, in all the aspects of the present invention where a glycerol kinase and/or a transketolase is overexpressed, the glycerol kinase is encoded by the GUT1 gene and/or the transketolase is encoded by the TKL1 gene.

The enzyme(s) overexpressed in said yeast strain can be an endogenous enzyme of said strain. The enzyme(s) overexpressed in said yeast strain can also be from any prokaryotic or eukaryotic organism. The coding sequence of the genes encoding this/these enzyme(s) can be optimized for its expression in the yeast by methods well known to those skilled in the art (for review, see Hedfalk, 2012).

The term overexpressing an enzyme in a yeast strain, herein refers to artificially increasing the quantity of said enzyme produced in a yeast strain compared to a reference (control) yeast strain wherein said enzyme is not overexpressed. This term also encompasses expression of an enzyme in a yeast strain which does not naturally contain a gene encoding said enzyme.

The glycerol kinase activity of an enzyme can be measured by quantifying formation of glyceroladehyde 3 phosphate from glycerol, as described in Sprague et al. (1977).

In yeasts, the glycerol kinase is encoded by the GUT1 gene. More particularly, the coding sequence of the GUT1 gene and the peptide sequence of the glycerol kinase of Y. lipolytica CLIB122 are available in the Génolevures or GenBank databases under the following accession numbers YALI0F00484g/YALI0F00484p (referred to as SEQ ID NO: 8).

The glycerol-3P dehydrogenase activity of an enzyme can be measured by quantifying the release of dihydroxyacetone phosphate from glycerol 3 phosphate, as described in Lindgren et al. (1977).

In yeasts, the glycerol-3P dehydrogenase is encoded by the GUT2 gene. More particularly, the coding sequence of the GUT2 gene and the peptide sequence of the glycerol-3P dehydrogenase of Y. lipolytica CLIB122 are available in the Génolevures or GenBank databases under the following accession numbers YALI0B13970g/YALI0B13970p (referred to as SEQ ID NO: 9).

The triose phosphate isomerase activity of an enzyme can be measured by quantifying the release of dihydroxyacetone phosphate from glyceraldehyde 3 phosphate, as described in Sharma et al. (2012).

In yeasts, the triose phosphate isomerase is encoded by the TIM1 gene. More particularly, the coding sequence of the TIM1 gene and the peptide sequence of the triose phosphate isomerase of Y. lipolytica CLIB122 are available in the Génolevures or GenBank databases under the following accession numbers YALI0F05214g/YALI0F05214p (referred to as SEQ ID NO: 10).

The transketolase activity of an enzyme can be measured by quantifying the formation of NAD+ from xylulose 5 phosphate, ribose 5 phosphate and NADH, as described in Matsushika et al. (2012).

In yeasts, the transketolase is encoded by the TKL1 gene. More particularly, the coding sequence of the TKL1 gene and the peptide sequence of the transketolase of Y. lipolytica CLIB122 are available in the Génolevures or GenBank databases under the following accession numbers YALI0E06479g/YALI0E06479p (referred to as SEQ ID NO: 11).

The erythrose 4 phosphate phosphatase activity of an enzyme can be measured by quantifying the formation of erythrose from erythrose 4 phosphate. It could also be screened by the detection of released phosphate (Pi) with the highly sensitive Malachite Green reagent as described in Baykov et al. (1988) or Kuznetsova et al. (2006).

The erythrose 4 phosphate phosphatase is encoded by an E4PK gene. The yeast gene coding for this enzyme has not been yet identified. However in bacteria, some proteins have shown to present erythrose 4 phosphate phosphatase activity. In Synechocys sp PCC6803 the erythrose 4 phosphate phosphatase is encoded by the sII1524 gene (Accession number WP_010873080 in the GeneBank database, International Application WO 2015/147644). In Thermotoga maritima MSB8 the erythrose 4 phosphate phosphatase is encoded by the TM1254 gene (Accession number NP 229059 in the GeneBank database, International Application WO 2015/147644). In Escherichia coli strain K12 the erythrose 4 phosphate phosphatase is encoded by the YidA gene (Accession number NP_418152 in the GeneBank database (Kuznetsova et al., 2006)).

The erythrose reductase activity of an enzyme can be measured by quantifying the formation of NADP+ from erythrose and NADPH, as described in Ishizuka et al. (1992).

In yeasts, the erythrose reductase is encoded by a gene belonging to the aldo-keto reductase family (AKR or ALR). The coding sequence of the AKR gene and the amino acid sequence of the erythrose reductase of Candida magnolia (ALR1) are available in the GenBank database under the following accession number FJ550210 (Lee et al., 2010, referred to as SEQ ID NO: 12).

The invertase activity of an enzyme can be measured by quantifying the release of reducing sugar from sucrose as described in Miller (1959).

A genetically modified Y. lipolytica strain comprising an invertase expression cassette composed of Saccharomyces cerevisiae Suc2p secretion signal sequence followed by the SUC2 sequence and under the control of the Y. lipolytica pTEF promoter is described in Lazar et al. (2013). The overexpression of invertase allows growth on sucrose-based raw materials.

Advantageously, the enzyme to overexpress is an endogenous enzyme of the mutated strain, provided that said strain naturally expresses the enzyme as defined above.

Overexpression of an enzyme as defined above—which can be an endogenous, ortholog or heterologous enzyme—in a yeast strain, in particular in a Yarrowia strain according to the present invention can be obtained in various ways by methods known per se.

Overexpression of an enzyme as defined in the present invention may be performed by placing one or more (preferably two or three) copies of the coding sequence (CDS) of the sequence encoding said enzyme under the control of appropriate regulatory sequences. Said regulatory sequences include promoter sequences, located upstream (at 5′ position) of the ORF of the sequence encoding said enzyme, and terminator sequences, located downstream (at 3′ position) of the ORF of the sequence encoding said enzyme.

Promoter sequences that can be used in yeast are well known to those skilled in the art and may correspond in particular to inducible or constitutive promoters. Examples of promoters which can be used according to the present invention, include the promoter of a Y. lipolytica gene which is strongly repressed by glucose and is inducible by the fatty acids or triglycerides such as the promoter of the PDX2 gene encoding the acyl-CoA oxidase 2 (AOX2) of Y. lipolytica and the promoter of the LIP2 gene described in International Application WO 01/83773. One can also use the promoter of the FBA1 gene encoding the fructose-bisphosphate aldolase (see Application US 2005/0130280), the promoter of the GPM gene encoding the phosphoglycerate mutase (see International Application WO 2006/0019297), the promoter of the YAT1 gene encoding the transporter ammonium (see Application US 2006/0094102), the promoter of the GPAT gene encoding the O-acyltransferase glycerol-3-phosphate (see Application US 2006/0057690), the promoter of the TEF gene (Müller et al., 1998; Application US 2001/6265185), the hybrid promoter hp4d (described in International Application WO 96/41889), the hybrid promoter XPR2 described in Mazdak et al. (2000) or the hybrid promoters UAS1-TEF or UAStef-TEF described in Blazeck et al. (2011, 2013, 2014).

Advantageously, the promoter is the promoter of the TEF gene.

Terminator sequences that can be used in yeast are also well known to those skilled in the art. Examples of terminator sequences which can be used according to the present invention include the terminator sequence of the PGK1 gene and the terminator sequence of the LIP2 gene described in International Application WO 01/83773.

The nucleotide sequence of the coding sequences of the heterologous genes can be optimized for expression in yeast by methods well known in the art (see for review Hedfalk, 2012).

Overexpression of an endogenous enzyme as defined above can be obtained by replacing the sequences controlling the expression of said endogenous enzyme by regulatory sequences allowing a stronger expression, such as those described above. The skilled person can replace the copy of the gene encoding an endogenous enzyme in the genome, as well as its own regulatory sequences, by genetically transforming the yeast strain with a linear polynucleotide comprising the ORF of the sequence coding for said endogenous enzyme under the control of regulatory sequences such as those described above. Advantageously, said polynucleotide is flanked by sequences which are homologous to sequences located on each side of said chromosomal gene encoding said endogenous enzyme. Selection markers can be inserted between the sequences ensuring recombination to allow, after transformation, to isolate the cells in which integration of the fragment occurred by identifying the corresponding markers. Advantageously also, the promoter and terminator sequences belong to a gene different from the gene encoding the endogenous enzyme to be overexpressed in order to minimize the risk of unwanted recombination into the genome of the yeast strain.

Overexpression of an endogenous enzyme as defined above can also be obtained by introducing into the yeast strain extra copies of the gene encoding said endogenous enzyme under the control of regulatory sequences such as those described above. Said additional copies encoding said endogenous enzyme may be carried by an episomal vector, that is to say capable of replicating in the yeast strain. Preferably, these additional copies are carried by an integrative vector, that is to say, integrating into a given location in the yeast genome, e.g., Yarrowia genome (Madzak et al., 2004). In this case, the polynucleotide comprising the gene encoding said endogenous enzyme under the control of regulatory regions is integrated by targeted integration. Said additional copies can also be carried by PCR fragments whose ends are homologous to a given locus of the yeast strain, allowing integrating said copies into the yeast genome by homologous recombination. Said additional copies can also be carried by auto-cloning vectors or PCR fragments, wherein the ends have a zeta region absent from the genome of the yeast, allowing the integration of said copies into the yeast genome, e.g., Yarrowia genome, by random insertion as described in Application US 2012/0034652.

Targeted integration of a gene into the genome of a yeast cell is a molecular biology technique well known to those skilled in the art: a DNA fragment is cloned into an integrating vector, introduced into the cell to be transformed, wherein said DNA fragment integrates by homologous recombination in a targeted region of the recipient genome (Orr-Weaver et al., 1981).

Methods for transforming yeast are also well known to those skilled in the art and are described, inter alia, by Ito et al. (1983), Klebe et al. (1983) and Gysler et al., (1990).

Any gene transfer method known in the art can be used to introduce a gene encoding an enzyme. Preferably, one can use the method with lithium acetate and polyethylene glycol described by Gaillardin et al., (1987) and Le Dall et al., (1994).

A preferred method for overexpressing an enzyme in a yeast strain comprises introducing into the genome of said yeast strain a DNA construct comprising a nucleotide sequence encoding said enzyme, placed under the control of a promoter.

Method for overexpressing genes in Yarrowia lipolytica is well known as described in example in Nicaud et al. (2002) and Nicaud (2012).

The overexpression of Y. lipolytica endogenous Y. lipolytica genes GUT1 GUT2, TKL1, and the heterologous Candida Magnoliae cmALR1 gene in a Y. lipolytica strain is reported in Carly et al., 2015.

The present invention also provides means for carrying out said overexpression.

This includes, in particular, recombinant DNA constructs for expressing at least one enzyme as defined above (GUT1, GUT2, TIM, TKL1, E4PK, ALR1, SUC2, EYD1) in a yeast cell, in particular in a Yarrowia cell. These DNA constructs can be obtained and introduced in said yeast strain by the well-known techniques of recombinant DNA and genetic engineering.

Recombinant DNA constructs of the invention include in particular expression cassettes, comprising a polynucleotide encoding at least one enzyme as defined above (i.e., a glycerol kinase, a glycerol-3P dehydrogenase, a triose isomerase, a transketolase, an erythrose 4 phosphate phosphatase, an erythrose reductase, an invertase, an erythritol dehydrogenase) preferably a glycerol kinase and/or a transketolase and/or an erythritol dehydrogenase, each polynucleotide encoding an enzyme being under the control of a promoter functional in a yeast cell as defined above.

The expression cassettes generally also include a transcriptional terminator, such as those describes above. They may also include other regulatory sequences, such as transcription enhancer sequences.

Recombinant DNA constructs of the invention also include recombinant vectors containing expression cassettes comprising a polynucleotide encoding at least one enzyme as defined above, each polynucleotide encoding an enzyme being under transcriptional control of a suitable promoter.

Recombinant vectors of the invention may also include other sequences of interest, such as, for instance, one or more marker genes, which allow for selection of transformed yeast cells.

The invention also comprises host cells containing a recombinant DNA construct of the invention. These host cells can be prokaryotic cells (such as bacteria cells) or eukaryotic cells, preferably yeast cells.

The invention also provides a method for obtaining a mutant erythritol-producing yeast strain, preferably a mutant Yarrowia strain, more preferably a mutant Y. lipolytica strain, having an enhanced erythritol productivity and/or yield (advantageously an enhanced erythritol productivity and yield) compared to the parent yeast strain, comprising inhibiting in the parent erythritol-producing yeast strain (of said mutant yeast strain) the expression or the activity of an endogenous L-erythrulose kinase (EYK; EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and optionally overexpressing in said yeast strain at least one enzyme selected from the group consisting of a glycerol kinase, a glycerol-3P dehydrogenase, a triose isomerase, a transketolase, an erythrose 4 phosphate phosphatase, an erythrose reductase and an invertase as defined above, preferably a glycerol kinase or a transketolase and more preferably a glycerol kinase and a transketolase.

Said overexpression can be obtained by transforming said yeast cell with at least one recombinant DNA constructs as defined above for expressing at least one enzyme selected from the group consisting of a glycerol kinase, a glycerol-3P dehydrogenase, a triose isomerase, a transketolase, an erythrose 4 phosphate phosphatase, an erythrose reductase and an invertase as defined above, preferably a glycerol kinase or a transketolase and more preferably a glycerol kinase and a transketolase.

More preferably, the method for obtaining a mutant erythritol-producing yeast strain, preferably a mutant Yarrowia strain, more preferably a mutant Y. lipolytica strain, having an enhanced erythritol productivity and/or yield (advantageously an enhanced erythritol productivity and yield) compared to the parent yeast strain, comprising inhibiting in the parent erythritol-producing yeast strain (of said mutant yeast strain) the expression or the activity of an endogenous L-erythrulose kinase (EYK; EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing in said yeast strain a glycerol kinase and a transketolase.

More advantageously, for obtaining a mutant erythritol-producing yeast strain, preferably a mutant Yarrowia strain, more preferably a mutant Y. lipolytica strain, having an enhanced erythritol productivity and/or yield (advantageously an enhanced erythritol productivity and yield) compared to the parent yeast strain, comprising inhibiting in the parent erythritol-producing yeast strain (of said mutant yeast strain) the expression or the activity of an endogenous L-erythrulose kinase (EYK; EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing in said yeast strain the glycerol kinase encoded by the GUT1 gene and the transketolase encoded by the TKL1 gene.

In one embodiment, the method for obtaining a mutant erythrulose-producing yeast strain, preferably a mutant Yarrowia strain, more preferably a mutant Y. lipolytica strain, having an enhanced erythrulose productivity and/or yield (advantageously an enhanced erythrulose productivity and yield) compared to the parent yeast strain, comprises inhibiting in the parent erythrulose-producing yeast strain (of said mutant yeast strain) the expression or the activity of an endogenous L-erythrulose kinase (EYK; EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and overexpressing in said yeast strain an erythritol dehydrogenase and optionally overexpressing in said yeast strain at least one enzyme selected from the group consisting of a glycerol kinase, a glycerol-3P dehydrogenase, a triose isomerase, a transketolase, an erythrose 4 phosphate phosphatase, an erythrose reductase and an invertase as defined above, preferably a glycerol kinase and/or a transketolase. Preferably said method comprises overexpressing the erythritol dehydrogenase encoded by the EYD1 gene and optionally the glycerol kinase encoded by the GUT1 gene and/or the transketolase encoded by the TKL1 gene.

Also in this aspect of the invention, the EYD1 gene is preferably of sequence SEQ ID NO: 7 (YALI_EYD1).

In another aspect, the present invention is also related to a method for obtaining a mutant erythritol-producing yeast strain, preferably a mutant Yarrowia strain, more preferably a mutant Y. lipolytica strain, having an enhanced erythritol productivity and/or yield (advantageously an enhanced erythritol productivity and yield) without production of erythrulose compared to the parent yeast strain, comprising inhibiting in the parent erythrulose-producing yeast strain (of said mutant yeast strain) the expression or the activity of an endogenous L-erythrulose kinase (EYK; EC 2.7.1.27) having at least 50% identity or by order of increasing preference at least 55%, 60%, 65%, 70%, 75%, 80%, 85%, 90%, 92%, 95%, 96%, 97%, 98% or 99% identity, with the polypeptide of sequence SEQ ID NO: 1 (YALI_EYK1) and inhibiting the expression or the activity of an endogenous erythritol dehydrogenase, preferably inhibiting the expression or the activity of the endogenous erythritol dehydrogenase of sequence SEQ ID NO: 7 (YALI_EYD1) or of an endogenous erythritol dehydrogenase having at least 50% identity or by order of increasing preference at least 55%, 60%, 65%, 70%, 75%, 80%, 85%, 90%, 92%, 95%, 96%, 97%, 98% or 99% identity, with the sequence SEQ ID NO: 7 (YALI_EYD1). Optionally said method further comprises overexpressing in said yeast strain at least one enzyme selected from the group consisting of a glycerol kinase, a glycerol-3P dehydrogenase, a triose isomerase, a transketolase, an erythrose 4 phosphate phosphatase, an erythrose reductase and invertase as defined above, preferably a glycerol kinase and/or a transketolase. Preferably said method comprises overexpressing the glycerol kinase encoded by the GUT1 gene and/or the transketolase encoded by the TKL1 gene.

In another aspect, the present invention is also related to a method for obtaining a mutant erythritol-producing yeast strain, preferably a mutant Yarrowia strain, more preferably a mutant Y. lipolytica strain, having an enhanced erythritol productivity and/or yield (advantageously an enhanced erythritol productivity and yield) without production of erythrulose compared to the parent yeast strain, comprising inhibiting the expression or the activity of an endogenous erythritol dehydrogenase, preferably inhibiting the expression or the activity of the endogenous erythritol dehydrogenase of sequence SEQ ID NO: 7 (YALI_EYD1) or of an endogenous erythritol dehydrogenase having at least 50% identity or by order of increasing preference at least 55%, 60%, 65%, 70%, 75%, 80%, 85%, 90%, 92%, 95%, 96%, 97%, 98% or 99% identity, with the sequence SEQ ID NO: 7 (YALI_EYD1). Also for this aspect of the invention, said method may optionally further comprise overexpressing at least one enzyme selected from the group consisting of a glycerol kinase, a glycerol-3P dehydrogenase, a triose isomerase, a transketolase, an erythrose 4 phosphate phosphatase, an erythrose reductase and invertase as defined above, preferably a glycerol kinase and/or a transketolase. Preferably said method comprises overexpressing the glycerol kinase encoded by the GUT1 gene and/or the transketolase encoded by the TKL1 gene.

The present invention also provides a mutant erythritol and/or erythrulose-producing yeast strain, preferably a Yarrowia strain, more preferably a Y. lipolytica strain, wherein the expression or the activity of the endogenous L-erythrulose kinase as defined above is inhibited and optionally wherein at least one enzyme selected from the group consisting of a erythritol dehydrogenase, a glycerol kinase, a glycerol-3P dehydrogenase, a triose isomerase, a transketolase, an erythrose 4 phosphate phosphatase, an erythrose reductase and an invertase as defined above, preferably a glycerol kinase or a transketolase, is overexpressed, and more preferably a glycerol kinase and a transketolase are overexpressed.

The present invention also provides a mutant erythritol-producing yeast strain, preferably a Yarrowia strain, more preferably a Y. lipolytica strain, wherein the expression or the activity of the endogenous L-erythrulose kinase as defined above is inhibited and optionally at least 1, 2, 3, 4, 5, 6 or the 7 enzymes selected from the group consisting of glycerol kinase, a glycerol-3P dehydrogenase, a triose isomerase, a transketolase, an erythrose 4 phosphate phosphatase, an erythrose reductase and an invertase as defined above, preferably at least a glycerol kinase or a transketolase are overexpressed.

More preferably, a glycerol kinase and a transketolase as defined above are overexpressed in the mutant erythritol-producing yeast strain, preferably a Yarrowia strain, more preferably a Y. lipolytica strain, wherein the expression or the activity of the endogenous L-erythrulose kinase as defined above is inhibited. More advantageously in this mutant, the glycerol kinase is encoded by the GUT1 gene and the transketolase is encoded by the TKL1 gene.

The present invention also provides a mutant erythrulose-producing yeast strain, preferably a Yarrowia strain, more preferably a Y. lipolytica strain, wherein the expression or the activity of the endogenous L-erythrulose kinase as defined above is inhibited and an erythritol dehydrogenase as defined above is overexpressed and optionally at least 1, 2, 3, 4, 5, 6 or the 7 enzymes selected from the group consisting of glycerol kinase, a glycerol-3P dehydrogenase, a triose isomerase, a transketolase, an erythrose 4 phosphate phosphatase, an erythrose reductase and an invertase as defined above, preferably at least a glycerol kinase or a transketolase, is overexpressed.

Even more preferably, a glycerol kinase and a transketolase as defined above are overexpressed in addition to the erythritol dehydrogenase as defined above, in the mutant erythrulose-producing yeast strain, preferably a Yarrowia strain, more preferably a Y. lipolytica strain, wherein the expression or the activity of the endogenous L-erythrulose kinase as defined above is inhibited. More advantageously in this mutant, the glycerol kinase is encoded by the GUT1 gene, the transketolase is encoded by the TKL1 gene and the erythritol dehydrogenase is encoded by the EYD1 gene.

The present invention also provides a mutant erythritol-producing yeast strain without production of erythrulose, preferably a Yarrowia strain, more preferably a Y. lipolytica strain, wherein the expression or the activity of the endogenous L-erythrulose kinase as defined above is inhibited and the expression or the activity of the endogenous erythritol dehydrogenase as defined above is inhibited and optionally at least 1, 2, 3, 4, 5, 6 or the 7 enzymes selected from the group consisting of glycerol kinase, a glycerol-3P dehydrogenase, a triose isomerase, a transketolase, an erythrose 4 phosphate phosphatase, an erythrose reductase and an invertase as defined above, preferably at least a glycerol kinase or a transketolase, is overexpressed.

Even more preferably, a glycerol kinase and a transketolase as defined above are overexpressed, preferably a Yarrowia strain, more preferably a Y. lipolytica strain, wherein the expression or the activity of the endogenous L-erythrulose kinase as defined above and of the endogenous erythritol dehydrogenase as defined above is inhibited. More advantageously in this mutant, the glycerol kinase is encoded by the GUT1 gene and the transketolase is encoded by the TKL1 gene.

The present invention also provides a mutant erythritol-producing yeast strain without production of erythrulose, preferably a Yarrowia strain, more preferably a Y. lipolytica strain, wherein the expression or the activity of the endogenous erythritol dehydrogenase (EC 1.1.1.9) as defined above is inhibited and optionally wherein at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21) and an invertase (EC 3.2.1.26), preferably a glycerol kinase and/or a transketolase, is overexpressed in said strain.

Said mutant yeast strain can be obtained by the method for obtaining a mutant erythritol and/or erythrulose-producing yeast strain as described above.

The mutant yeast strain of the invention includes not only the yeast cell resulting from the initial mutagenesis or transgenesis, but also their descendants, as far as the expression or the activity of the endogenous L-erythrulose kinase is inhibited and optionally as far as at least one enzyme selected from the group consisting of a glycerol kinase, a glycerol-3P dehydrogenase, a triose isomerase, a transketolase, an erythrose 4 phosphate phosphatase, an erythrose reductase and an invertase as defined above, preferably a glycerol kinase and/or a transketolase, is overexpressed.

The present invention also provides a mutant erythritol and/or erythrulose-producing yeast strain, preferably a Yarrowia strain, more preferably a Y. lipolytica strain, wherein the expression or the activity of the endogenous L-erythrulose kinase as defined above is inhibited and optionally further comprising, stably integrated in its genome, at least one recombinant DNA constructs for expressing at least one enzyme selected from the group consisting of a glycerol kinase, a glycerol-3P dehydrogenase, a triose isomerase, a transketolase, an erythrose 4 phosphate phosphatase, an erythrose reductase, an invertase and an erythritol dehydrogenase as defined above, preferably a glycerol kinase and/or a transketolase and/or an erythritol dehydrogenase as defined above, and even more preferably the glycerol kinase encoded by the GUT1 gene and/or the transketolase encoded by the TKL1 gene and/or an erythritol dehydrogenase encoded by the EYD1 gene.

Similar embodiments relating to a mutant erythritol-producing yeast strain without production of erythrulose, preferably a Yarrowia strain, more preferably a Y. lipolytica strain, wherein the expression or the activity of the endogenous L-erythrulose kinase as defined above is inhibited, wherein the expression or the activity of the endogenous erythritol dehydrogenase as defined above is inhibited and optionally comprising, stably integrated in its genome, at least one recombinant DNA constructs for expressing at least one enzyme as defined above, are also provided by the present invention. As well as those relating to a mutant erythritol-producing yeast strain without production of erythrulose, preferably a Yarrowia strain, more preferably a Y. lipolytica strain, wherein the expression or the activity of the endogenous erythritol dehydrogenase as defined above is inhibited and optionally comprising, stably integrated in its genome, at least one recombinant DNA constructs for expressing at least one enzyme as defined above.

The present invention also provides the use of a mutant yeast strain, preferably a mutant Yarrowia strain, more preferably a mutant Y. lipolytica strain, of the invention for producing erythritol and/or erythrulose.

The present invention also provides the use of a mutant yeast strain, preferably a mutant Yarrowia strain, more preferably a mutant Y. lipolytica strain, of the invention for bioconverting erythritol to erythrulose.

The method for enhancing the erythrulose productivity and/or yield of an erythrulose-producing yeast strain according to the present invention can further comprise a step of culturing said erythrulose-producing yeast strain at a biomass comprised between 1 g and 150 g CDW/L, preferably between 10 g and 50 g CDW/L, in a medium comprising an erythritol concentration comprised between 1 g/L and 200 g/L, preferably between 10 g/L and 80 g/L.

The present invention also provides a method for producing erythritol and/or erythrulose, comprising a step of growing a mutant yeast strain, preferably a mutant Yarrowia strain, more preferably a mutant Y. lipolytica strain, of the invention.

The present invention also provides a method for producing erythrulose or bioconverting erythritol to erythrulose, comprising a step of growing a mutant yeast strain, preferably a mutant Yarrowia strain, more preferably a mutant Y. lipolytica strain, of the invention, at a biomass comprised between 1 g and 150 g CDW/1, preferably between 10 g and 50 g CDW/1, in a medium comprising an erythritol concentration comprised between preferably between 1 g/L and 200 g/L, more preferably between 10 g/L and 80 g/L.

Methods for extracting and purifying erythritol produced by cultured yeast strains are well known to those skilled in the art, e.g., patent application EP 0 845 538; Rymowicz et al., 2008; Moon et al., 2010; Tomaszewska et al., 2012; Mirończuk et al., 2014.

Method for purifying erythrulose is described in Morii et al. (1985). HPLC method for erythrulose quantification is described in Ge et al. (2012).

NMR method for identifying erythritol and erythrulose are described in Nishimura et al., (2006) and Hirata et al. (1999).

The mutant yeast of the invention can be cultured in repeated batch, fed-batch on continuous cultures as planktonic cell or biofilm (i.e., cell growing on the surface or inside a solid support).

Advantageously, the source of carbon can be glycerol, glucose, sucrose, xylose, molasses, preferably glycerol.

The present invention will be understood more clearly from the further description which follows, which refers to non-limitative examples illustrating the inhibition of the expression of the YALI0F01606g gene encoding EYK1 of SEQ ID NO: 1 in Y. lipolytica, as well as to the appended.

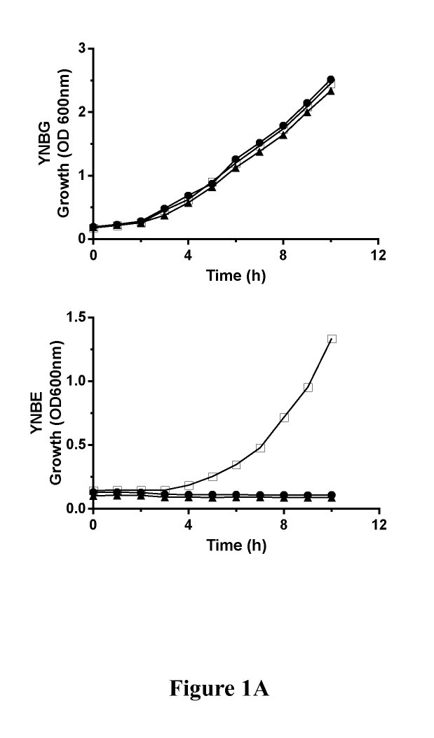

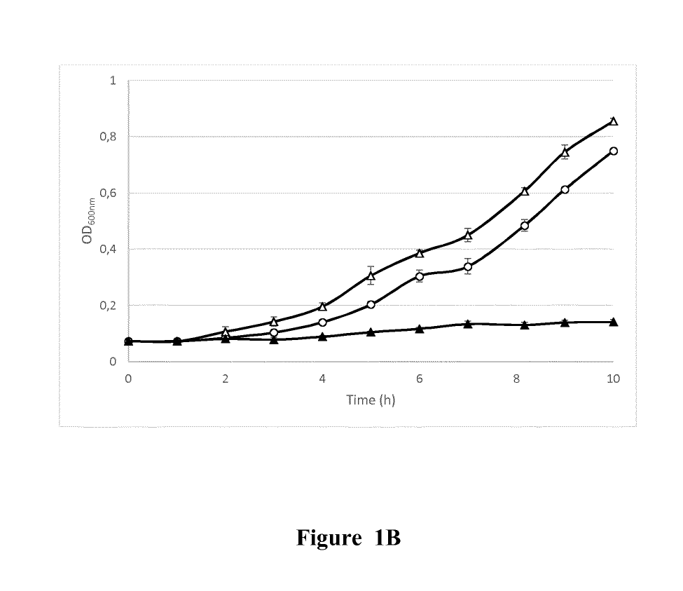

FIG. 1. Panel A shows the growth curve of Y. lipolytica strain W29 (□; empty square) JMY4949 (●; filled circle) and FCY001 (▴; filled triangle) during shake-flask culture in minimal YNBG and YNBE medium. Panel B shows the growth curve of Y. lipolytica strain W29 on medium YNBG (◯; empty circle), RIY208 on medium YNBG (Δ; open triangle) and RIY208 on medium YNBE (▴; filled triangle). Cultures were performed in shake flask.

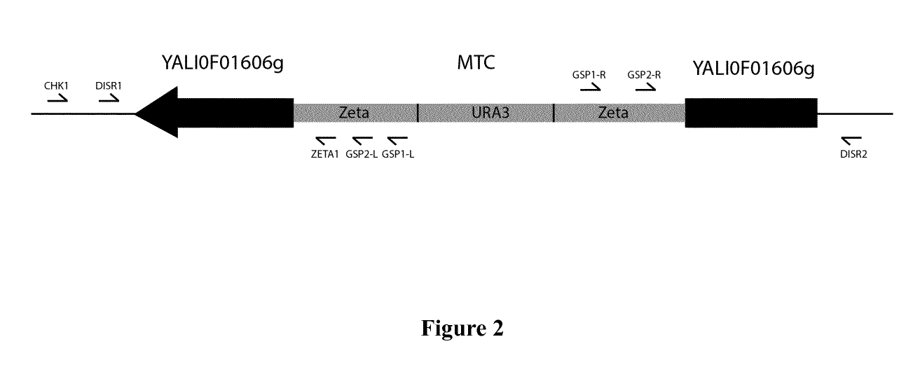

FIG. 2 shows the schematic representation of the insertion locus of the mutagenesis cassette (MTC, grey) in the YALI0F01606 gene (black) in the JMY4949 genome. Primers are indicated by the small arrow.

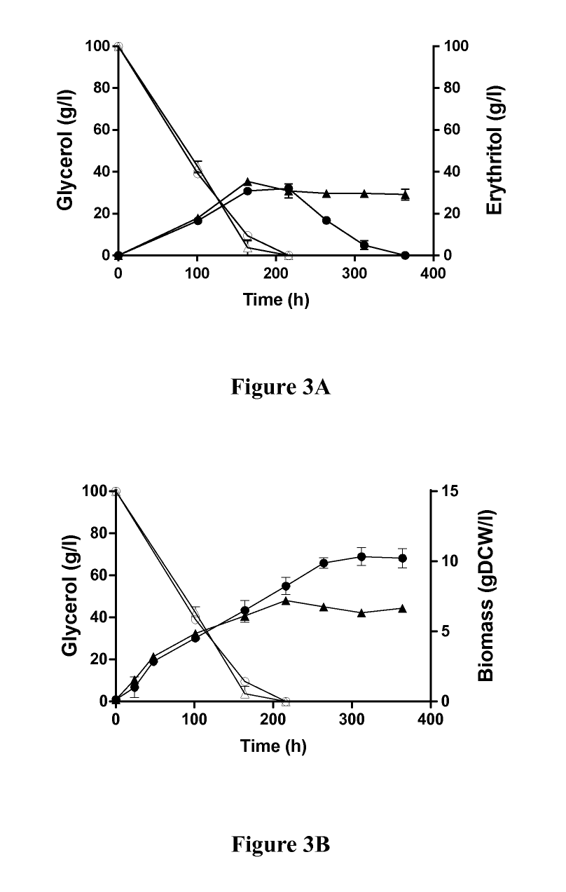

FIG. 3 shows the glycerol and erythritol concentration in the culture medium (A) and cell growth (B) during shake-flask culture of erythritol production from W29 and FCY001. Panel A: ∘ (empty circle): glycerol (W29); Δ (empty triangle): glycerol (FCY001); ● (filled circle): erythritol (W29); Δ(filled triangle): erythritol (FCY001). Panel B: ∘ (empty circle): glycerol (W29); Δ (empty triangle): glycerol (FCY001); ● (filled circle): biomass W29; ▴ (filled triangle): biomass FCY001.

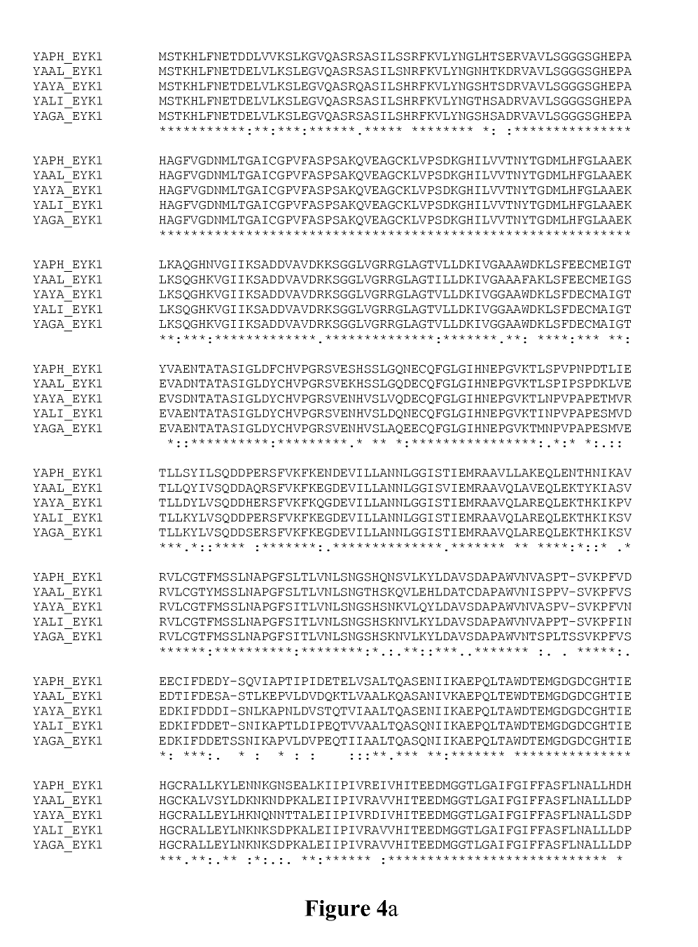

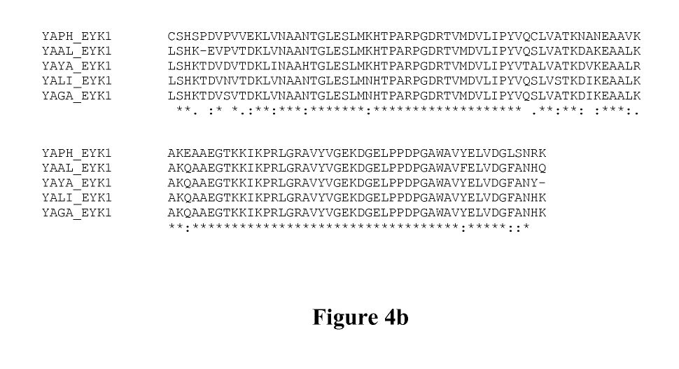

FIG. 4 shows the CLUSTAL multiple sequence alignment of EYK1 genes in the Yarrowia Glade performed by MUSCLE (3.8). Sequences are from strains YALI: Yarrowia lipolytica CLIB122 (100%); YAGA: Yarrowia galli CBS 9722 (96.77%); YAYA: Yarrowia yakushimensis CBS 10253 (91.62%); YAAL: Yarrowia alimentaria CBS 10151 (87.22%) and YAPH: Yarrowia phangnensis CBS 10407 (85.01%). Maximal identities with Yarrowia lipolytica EYK1 are indicated in brackets.

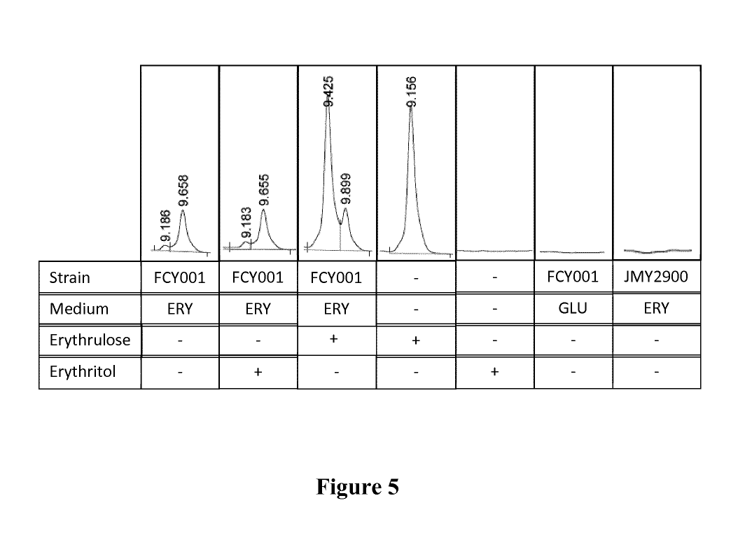

FIG. 5 shows the HPLC analysis of culture supernatant of strains FCY001 and JMY2900 grown in YNBcasa containing 10 g/l of erythritol (ERY) or glucose (GLU). Chromatograms correspond to the U.V. signal recorded at 210 nm between 9 and 10 min of analysis. Samples were analysed in the presence (+) or in absence (−) of polyol standards at a final concentration of 2 g/L.

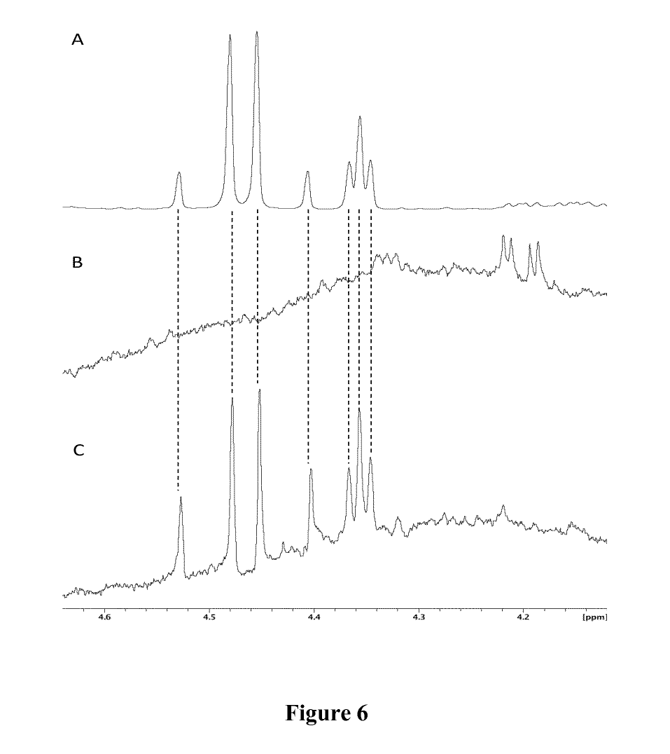

FIG. 6 shows NMR spectra of culture supernatants of strain W29 and FCY001. A: Erythrulose solution at 2 g/L in D₂O. B: Culture supernatants of the Y. lipolytica wild-type strain W29. C: Culture supernatants of strain FCY001.

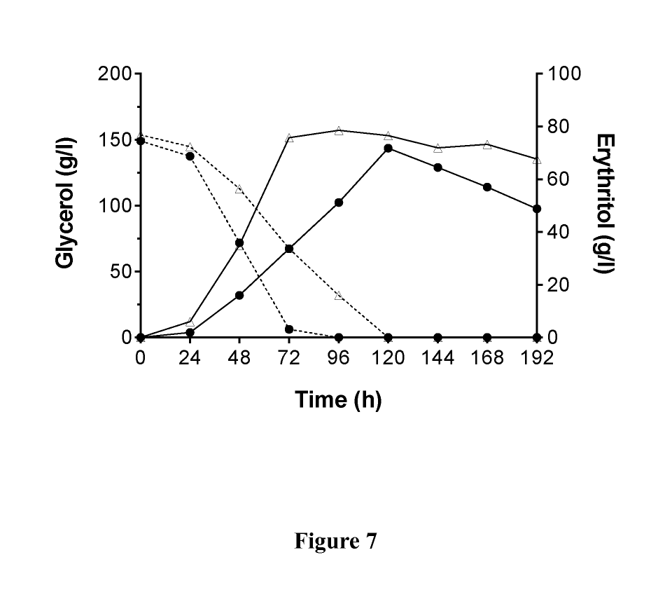

FIG. 7 shows erythritol production (plain line) and glycerol consumption (doted line) for FCY218 (GUT1-TKL1-Δeyk, triangle) and JMY2900 (WT, circle) during culture in bioreactor in EPB medium.

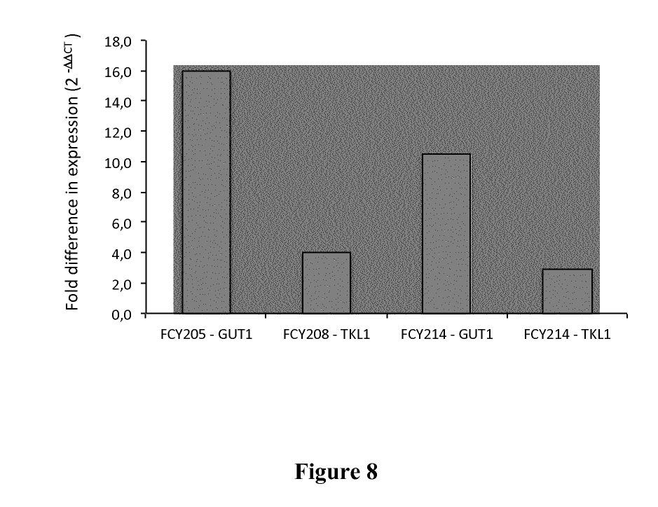

FIG. 8 shows relative expression of the genes GUT1 and TKL1 in strain FCY205, FCY208 and FCY214. The expression levels were standardized relative to the expression of the actin gene (ΔC_{T}); then the fold difference was calculated (2^{-ΔΔCT}) based on baseline expression in the wild type strain W29.

### EXAMPLES

1) Material and Methods

1.1) Strains and Media

Wild-type Y. lipolytica strains used in this study are:
W29 (MATa; Ery+) (Barth and Gaillardin, 1996)
Po1d (MATa ura3-302, leu2-270 xpr2-322; Ura−, Leu−, Ery+) (Barth, Gaillardin, 1996)
JMY2900, prototrophe derivative of Po1d used as WT control, (MATa ura3-302, leu2-270 xpr2-322; Ura+, Leu+, Ery+; Po1d, Ura+, Leu+) (Ledesma-Amaro et al., 2015)
JMY2101 (Leu+ derivative of Po1d, MATa ura3-302, xpr2-322; Ura−, Leu+, Ery+) (Leplat et al., 2015)
JMY4174 (MATa ura3-302 leu2-270 xpr2-322 Δdga1, Δlro1, Δpox1-6, LEU2; Ura− Leu+, Ery+)

Standard YPD and YNB media used for growth and transformation of Y. lipolytica were as described elsewhere (Fickers et al., 2003). YNBG and YNBE used for mutant screening consisted of YNB medium with glucose replaced respectively by 1% (w/v) glycerol or 1% (w/v) erythritol. For erythritol production, media used were based on Tomaszewska et al. (2012). Growth medium (EG) consisted of (per liter): glycerol 50 g; peptone 5 g; yeast extract 5 g. Production medium used for shake-flasks cultures (EPF) was (per liter): glycerol 100 g; yeast extract 1 g; NH₄Cl 4.5g; CuSO₄ 0.7×10⁻³ g; MnSO₄. H₂O 32×10⁻³ g; 0.72 M phosphate buffer at pH 4.3. Production medium for bioreactor production (EPB) was (per liter): glycerol 150 g; NH₄Cl 2 g; KH₂PO₄ 0.2 g; MgSO4×7 H₂O 1 g; yeast extract 1 g; NaCl 25 g.

Other Y. lipolytica strains used herein are the following:
JMY4949 (JMY4174 derivative, YALI0F01606::MTC-URA3); MATa ura3-302 leu2-270 xpr2-322 Δdga1, Δfro1, Δpox1-6, LEU2 YALI0F01606::MTC-URA3; Ura+ Leu+, Ery−);
FCY001 (JMY2101 derivative, YALI0F01606::MTC-URA3), MATa ura3-302, xpr2-322 YALI0F01606::MTC-URA3; Ura+, Leu+, Ery−;
RIY208 (JMY2101 derivative, Δeyk1::URA3), MATa ura3-302, xpr2-322 Δeyk1::URA3; Ura+, Leu+, Ery−;
RIY203 (Po1d, Δeyk), MATa ura3-302 leu2-270 xpr2-322 Δeyk1; Ura−, Leu−, Ery−;
FCY205 (Po1d, LEU2ex-pTEF-GUT1, URA3ex), MATa ura3-302 leu2-270 xpr2-322 LEU2ex-pTEF-GUT1, URA3ex, Ura+, Leu+, Ery+;
FCY208 (Po1d, URA3ex-pTEF-TKL1, LEU2), MATa ura3-302 leu2-270 xpr2-322 URA3ex-pTEF-TKL1, LEU2, Ura+, Leu+, Ery+
FCY214 (Po1d, LEU2ex-pTEF-GUT1, URA3ex-pTEF-TKL1), MATa ura3-302 leu2-270 xpr2-322 LEU2ex-pTEF-GUT1 URA3ex-pTEF-TKL1, URA3ex, Ura+, Leu+, Ery+
FCY218 (Po1d, Δeyk, LEU2ex-pTEF-GUT1, URA3ex-pTEF-TKL1), MATa ura3-302 leu2-270 xpr2-322 LEU2ex-pTEF-GUT1 URA3ex-pTEF-TKL1, Ura+, Leu+, Ery−
RIY146 MATa ura3-302 leu2-270 xpr2-322 Δeyk1::LEU2
RIY210 (RIY145, LEU2), MATa ura3-302 leu2-270 xpr2-322 Δeyk1::LEU2 URA3ex-pTEF-EYK1; Ura+, Leu+, Ery−;

1.2) Culture Conditions

All shake-flask cultures were performed at 28° C. in 250 mL flasks containing 50 mL of appropriate medium. Shake-flasks mutant screening cultures were carried in YNBE or YNBG for 11 h at 190 RPM after a 24 h YPD growth. Erythritol productions were carried in EPF medium for 10 days at 250 RPM after a 72 h EG growth. All cultures were performed in triplicates.

Bioreactors cultures were performed in 2-1 bioreactors (Biostat B-Twin, Sartorius) containing 1 L EPB medium at 28° C. for 96 h, after a 72 h EG growth. Stirrer speed was set at 800 RPM and aeration rate was kept at 1 vvmin⁻¹. pH was set at 3.0 and automatically adjusted by the addition of 20% (w/v) NaOH or 40% (w/v) H₃PO₄. Bioreactor cultures were performed in duplicates.

1.3) Analytical Methods

Cell growth was monitored by optical density at 600 nm (OD600) and dry cell weight (DCW) was calculated either from OD600 according to gDCW=OD600 nm/4.7 or based on the biomass according to gDCW=OD600 nm*0.29. Glycerol, erythritol and erythrulose concentrations in the media were determined by isocratic UV-RID-HPLC (Agilent 1100 series, Agilent Technologies) using an Aminex HPX-87H ion-exclusion column (300×7.8 mm Bio-Rad, Hercules, USA) with 15 mM Trifluoroacetic acid as mobile phase at a flow rate of 0.6 ml·min⁻¹ at 65° C. Samples were analyzed using refractive index and absorbance at a wavelength of 205 nm. Compounds were identified on the basis of the retention time using commercially available standards. Glycerol concentration was calculated from HPLC chromatogram based on the following calibration equations: glycerol concentration=[(pic area−1888)/66307] or glycerol concentration=[(pic area−1879)/76916].

1.4) General Molecular Biology Techniques

Standard molecular biology techniques were used (Green et al., 2012). Transformation and genetic manipulations of Y. lipolytica were done according to Barth and Gaillardin (1996). Genomic DNA from Y. lipolytica was prepared according to Querol et al., (1992). PCR reactions were performed on a MJ Mini Gradient Thermal Cycler (Bio-Rad) using DreamTaq DNA polymerase (Thermo Scientific), except for genome walking PCR (see below). 25 cycles were carried for each PCR reaction, and were as follows: denaturation at 95° C. for 30 s, annealing at 56° C. for 30 s, extension at 72° C. for 1 min/kb. A final 10 min extension was added as the last step. PCR fragments were purified from agarose gels using GeneJet Gel Extraction Kit (Thermo Scientific).

1.5) Mutant Library Screening

A library of randomly generated Y. lipolytica mutants was constructed by inserting a mutagenesis cassette (MTC) in the genome of the Y. lipolytica wild-type strain JMY4174 (Ura−). The MTC sequence consisted of two zeta regions from Ylt1 retrotransposon, allowing random genome insertion (Barth and Gaillardin 1996), flanking the URA3 gene for selection. 11,000 mutants were obtained and screened at the PICT-Genotoul Platform (INSA-Toulouse). After two growth phases on liquid YNB with 2% and 0.2% glucose concentrations respectively, the mutants were screened on two different solid media, YNBG and YNBE.

Colonies exhibiting normal growth on glycerol but slow growth on erythritol were selected for a second screening. After further growth on YNB, two replicates of each selected mutant were transferred on new plates containing YNBG or YNBE. The clones still showing a slow growth on erythritol for both replicates were selected for shake-flask screening, as described above.

1.6) Genome Walking

The insertion site of the MTC in JMY4949 strain was identified by genome walking using Universal GenomeWalker 2.0 (ClonTech Laboratories inc.). After extraction, genomic DNA was digested with four different restriction enzymes (DraI, EcoRV, PvuII, StuI) and the resulting fragments were ligated with the GenomeWalker adaptors. PCR reactions were performed on the ligated fragments using primers matching the adaptor (AP1, see Table 1) and either the 5′ side (GSP1-L) or the 3′ side (GSP1-R) of the MTC. This allowed to amplify only the genomic fragments containing the MTC and its surroundings.

A second PCR reaction with different primers (AP2 and either GSP1-L or GSP1-R) was then performed to ensure specificity. The PCR steps were performed using Advantage 2 Polymerase (ClonTech Laboratories inc.) and cycles were designed as recommended by the user manual. The resulting amplified fragments were separated by gel electrophoresis, purified, and sequenced with Sanger sequencing (GATC Biotech). A BLAST analysis of the sequences was then performed at the GREC site (http://gryc.inra.fr/) on the Y. lipolytica genome to identify the insertion site of the MTC.

1.7) Disruption of YALI0F1606g in a Wild-Type Strain

Construction of the FCY001 strain was achieved by disrupting the YALI0F01606g gene within JMY2101 strain. A 3700 base pairs (bp) region consisting of the MTC insertion site and its surroundings (1000 bp on each side of the MTC insertion site) was amplified from JMY4949 strain, using primers DISR1 and DISR2. The amplified fragment was analyzed by gel electrophoresis and purified. This fragment contained all the elements for a disruption cassette of YALI0F01606g; specific sequences for homologous recombination and site-directed insertion, and a selection marker (URA3 gene within the MTC). This purified disruption cassette was used to transform JMY2101 strain. Transformed strains were selected on YNB plates, and the success of the gene disruption was verified by PCR, using ZETA1 and CHK1 primers.

Strain RIY208 was constructed by disrupting the EYK1 gene in strain JMY2101 as described hereinafter. The EYK1 P and T fragments were amplified from strain W29 genomic DNA using primer pairs EYK1-PF/EYK1-PR and EYK1-TF/EYK1-TR, respectively. The URA3 marker was amplified from the JMP113 plasmid (Fickers et al. 2013) using the primer pair LPR-F/LPR-R. Primer EYK1-PR, EYK1-TR, LPR-F and LPR-R were designed to introduce an SfiI restriction site in amplified fragment. Amplicons were digested with SfiI before being purified and ligated, using T4 DNA ligase, at a molar ratio of 1:1. The ligation products were amplified via PCR using the primer pair EYK1-PF/EYK1-TR. They were then purified and used to transform strain JMY2101, this process yielded strain RIY208 (A eyk1::URA3). The prototroph derivative of strain RIY208, namely RIY203 was obtained according to Fickers et al. 2003.

Strain RIY203 was constructed using the same disruption cassette except that the transformed strain was Po1d. This process yielded strain RIY203.

1.8) Strain Construction for Overexpression of Glycerol Kinase and Transketolase

The different genes that were over-expressed are YALI0F00484g (GUT1, Glycerol kinase, Y. lipolytica; BamHI site removal) and YALI0E06479g (TKL1, Transketolase Y. lipolytica; Intron removal, ClaI site removal). Yeast genes were amplified from genomic DNA of strain Y. lipolytica W29.

Primers for gene amplification were designed to introduce an AvrII site at the 3′ end and a BamHI restriction sites at the 5′ end of genes YALI0F00484g and YALI0E06479g (Table 1). Introns and undesirable restriction sites were removed by overlap extension PCR and site-directed mutagenesis (Higuchi et al., 1988): BamHI site removal in YALI0F00484g (GUT1, Glycerol kinase, Y. lipolytica) was performed with primer GUT1F1/GUT1R1 (PCR1) and GUT1F2/GUT1F1 (PCR2) and finally with GUT1F1/GUT1F1 using amplicons from PCR1 and PCR2 as templates. Intron removal, ClaI site removal for YALI0E06479g (TKL1, Transketolase Y. lipolytica.) was performed using primer pairs TKLIF1/TKL1R1 (PCR1), TKL1F2/TKL1R2 (PCR2) and TKL1F3/TKL1R3, (PCR3). Finally, the modified TKL1 was amplified with primers TKLF1/TKL1R3 and amplicons from PCR1, PCR2 and PCR3 as template.

Amplicons were purified from agarose gel, before being digested using BamHI/AvrII restriction enzymes. The corresponding fragments were finally cloned into BamHI/AvrII digested JMP1047 (Lazar et al 2013) or JMP2563 (Dulermo et al 2017) vectors in order to obtain URA3 or LEU2 counterpart, respectively. The correctness of the resulting construct was verified by DNA sequencing.

Expression cassettes for genes GUT1 and TKLI were rescued from corresponding vectors by NotI digestion and purified from agarose gel before being used to transform Y. lipolytica strains Po1d or RIY203. Transformants were selected on YNB medium supplemented with uracil or leucine depending on their auxotrophy. Correctness of the constructed strain was verified by analytical PCR on genomic DNA using primer pairs URA3F/61stop or LEU2F/61stop, depending on the auxotrophic marker used for transformation. Prototrophic stains were obtained according to Fickers et al. 2003.

TABLE 1
Primers used for genome walking and strain constructions (Restriction sites are
underlined, mismatched bases for site-directed mutagenesis are in bold,
overhangs for overlap extension PCR are in italics) are the following:

| | | SEQ ID |
|---|---|---|
| Primer | Sequence (5′-3′) | No. |
| GSP1-L | TCTCGGTGGTCAATGCGTCAGAAGATATC | 13 |
| GSP2-L | AGCCGAGTGAATGTTGCCTGCCGTTAGT | 14 |
| GSP1-R | AGCGTTCGCCAATTGCTGCGCCATCGT | 15 |
| GSP2-R | ACACTACCGAGGTTACTAGAGTTGGGAAA | 16 |
| AP1 | GTAATACGACTCACTATAGGGC | 17 |
| AP2 | ACTATAGGGCACGCGTGGT | 18 |
| DISR1 | TGTAGCACCTGGGTCAACATTT | 19 |
| DISR2 | TCCGATGACCTGACTAGTGCG | 20 |
| CHK1 | GATTGCTCCGTTTGTAAGTACA | 21 |
| ZETA1 | TGGTCCTGTTCCACCTGAAC | 22 |
| GUT1 F1 | GACGGATCCATGTCTTCCTACGTAGGAGCTCTC (restriction site | 23 |
| | BamHI) | |
| GUT1 R1 | GTTATCCAGAATCCATCGGAC | 24 |
| GUT1 F2 | GGTCCGATGGATTCTGGATA | 25 |
| GUT1 R2 | GACCCTAGGTTACTCAAGCCAGCCAACAG (restriction site AvrII) | 26 |
| TKL1 F1 | CGAGGATCCATGGCTCCCCAATTTTCAAAG (restriction site | 27 |
| | BamHI) | |
| TKL1 R1 | GCCACAGCATCAATGCCAAGGTTCGGATGGTGTT | 28 |
| TKL1 F2 | ATCAACACCATCCGAACCTTGGCTATTGATGCTGTGGCCAAGGC | 29 |
| TKL1 R2 | GTTCTTGAGATCATCAATAGTGATGTCGTAGC | 30 |
| TKL1 F3 | GCTACGACATCACTATTGATGATCTCAAGAAC | 31 |
| TKL1 R3 | GACCCTAGGTTAGACACCGTGGCCGGGTC (restriction site AvrII) | 32 |
| URA3 F | AGGAAGAAACCGTGCTTAAGAG | 33 |
| LEU2 F | TAAGTCGTTTCTACGACGCATT | 34 |
| 61 Stop | GTAGATAGTTGAGGTAGAAGTTG | 35 |
| EYK-PF | GTTGTGTGATGAGACCTTGGTGC | 36 |
| EYK-PR | AAAGGCCATTTAGGCCGCAGCTCCTCCGACAATCTTG (restriction | 37 |
| | site SfiI) | |
| EYK-TF | TAAGGCCTTGATGGCCACAAGTAGAGGGAGGAGAAGC | 38 |
| | (restriction site SfiI) | |
| EYK-TR | GTTTAGGTGCCTGAAGACGGTG | 39 |
| LPR-F | ATAGGCCTAAATGGCCTGCATCGATCTAGGGATAACAGG | 40 |
| | (restriction site SfiI) | |
| LPR-R | ATAGGCCATCAAGGCCGCTAGATAGAGTCGAGAATTACCCTG | 41 |
| | (restriction site SfiI) | |
| GUT1-L-q | CCCTGTCCACCTACTTTGCC (target gene GUT1) | 42 |
| GUT1-R-q | TTGGAGGTGTCGGTGATGTG (target gene GUT1) | 43 |
| TKL1-P-L-q | CAGCAACACAGATGGCAACC (target gene GUT1 TKL1) | 44 |
| TKL1-T-R-q | CGAGACCTCCGCTGCTTACTAC (target gene GUT1 TKL1) | 45 |
| ACT-F | GGCCAGCCATATCGAGTCGCA (target gene ACT) | 46 |
| ACT-R | TCCAGGCCGTCCTCTCCC (target gene ACT) | 47 |

1.9) RNA Isolation and Transcript Quantification.

Shake-flask cultures were grown in EPF medium for 24 h. Cells were then collected and store at −80° C. RNA extraction and cDNA synthesis were performed as previously described (Sassi et al 2016). Primers for RT-qPCR are listed in Table 1. The results were normalized to actin gene and analyzed to the ddCT method (Sassi et al 2016). Samples were analyzed in duplicates.

2) Results

2.1) Mutant Screening

In order to isolate a Y. lipolytica strain unable to grow on erythritol, a library of 11,000 insertion mutants was screened on glycerol and erythritol medium plates. After the first screening, 188 mutants were selected for having a have normal growth on glycerol but a slow growth on erythritol. After a second screening, 10 mutants were still displaying this phenotype consistently and were selected for shake-flask screening. Among these, one mutant was confirmed to be deficient for erythritol consumption (FIG. 1A). No growth on erythritol was observed for this mutant, while it grew as fast as W29 on glycerol. This strain was named JMY4949.

2.2) Identification of the Disrupted Gene

In order to find which gene was disrupted in the JMY4949 strain, a genome walking analysis was performed. Primers designed to match the MTC allowed to amplify the region surrounding its insertion site in the JMY4949 genome. After sequencing this region, BLAST analysis revealed that the MTC insertion site was located within the YALI0F01606g gene, indicating that the disruption of this gene caused the loss of the ability to grow on erythritol (FIG. 2).

2.3) Construction of a Y. lipolytica Strain Disrupted in YALI0F1606g Gene

A disruption cassette of this gene YALI0F01606g was constructed to transform the wild-type strain JMY2101. The strain FCY001 was obtained as a result. This strain has the same genotype as W29 except for the disruption of YALI0F01606g. This strain was evaluated in shake-flasks in YNBG and YNBE medium, and exhibited the same phenotype as JMY4949 strain (FIG. 1A). These results confirmed that the YALI0F01606g gene is essential in the erythritol catabolism pathway, and that the disruption of this gene alone is sufficient to remove the ability of Y. lipolytica to use erythritol as a carbon source. In addition, growth of FCY001 did not show any growth defect on glycerol media (FIG. 1A). As shown in FIG. 1B, strain RIY208 which is also a strain disrupted in YALI0F1606g gene, shows a growth defect on YNBE medium. It showed a similar growth profile as compared to strain W29 on YNBG medium.

2.4) Shake-Flask Erythritol Production

In order to assess the effects of a ΔYALI0F01606g strain on erythritol production, shake-flask production cultures were carried using W29 and FCY001 (FIG. 3). After 7 days of culture and near glycerol exhaustion, FCY001 had produced 35.7 g/l erythritol while W29 had only produced 30.7 g/l, meaning that the disruption of YALIF01606g gene had a positive effect on erythritol production. Results also showed that as soon as glycerol was depleted, W29 strain began to use erythritol for its growth, leading to a quick decrease of erythritol concentration in the medium. On the other hand, only a small decrease in erythritol concentration was observed in the FCY001 culture, after which its concentration remained stable during at least seven days. The small drop in erythritol concentration might be due to a partial conversion of erythritol into L-erythrulose, which couldn't be further converted. This would be consistent with the hypothesis that YALI0F01606g is an EYK.

2.5) Bioreactor Erythritol Production

Batch bioreactor cultures of FCY001 and W29 were performed to further evaluate the benefits of a YALI0F01606g disruption in production conditions. Results are displayed in Table 2.

TABLE 2
Characteristic parameter of erythritol production during culture in
bioreactor of W29 and FCY001

| | strain | |
|---|---|---|
| Parameters | FCY001 | W29 |
| Yield (g · g⁻¹)* | 0.46 ± 0.15 | 0.34 ± 0.02 |
| Yield (g · g⁻¹)^{$} | 0.49 ± 0.02 | 0.39 ± 0.01 |
| Erythritol productivity (g · l⁻¹ · h⁻¹) | 0.59 ± 0.03 | 0.52 ± 0.05 |
| Specific erythritol productivity | 0.115 ± 0.005 | 0.089 ± 0.002 |
| (g · l⁻¹ · h⁻¹ · DCW⁻¹) ¤ | | |
| Specific glycerol uptake rate | 0.291 ± 0.013 | 0.253 ± 0.005 |
| (g · l⁻¹ · h⁻¹ · DCW⁻¹) ¤ | | |
| Specific erythritol productivity | 0.052 ± 0.005 | 0.040 ± 0.002 |
| (g · g_{DCW}⁻¹ · h⁻¹) * | | |
| Specific glycerol uptake rate | 0.110 ± 0.003 | 0.101 ± 0.003 |
| (g · g_{DCW}⁻¹ · h⁻¹) * | | |

*glycerol concentration was calculated according to glycerol concentration = [(pic area − 1888)/66307].
^{$}glycerol concentration was calculated according to glycerol concentration = [(pic area − 1879)/76916].
¤ specific productivity according to gDCW = OD600 nm/4.7
* specific productivity according to gDCW = OD600 nm*0.29

Bioreactor experiments confirmed the observations from the shake-flasks observations. Compared to W29, FCY001 had 25 to 35% higher yield depending on the method used for glycerol calculation, 28 to 30% higher specific productivity depending on the calculation method used for the conversion of the measured OD, and a 13% higher productivity. The significantly higher yield compared to the W29 strain might indicate that in a wild-type strain, some of the produced erythritol is consumed even before glycerol depletion. More surprising is the observation that FCY001 glycerol uptake is consistently faster than for W29, although its growth is slightly slower (data not shown), which would indicate that a YALI0F01606g disruption improves glycerol uptake, and that this increased glycerol uptake is mostly directed towards erythritol production rather than biomass production. These results altogether show that a YALI0F01606g disruption allows the improvement erythritol production while helping to keep its concentration stable after glycerol depletion.

2.6) Shake-Flask Erythrulose Production

In order to further assess the effects of the disruption of YALI0F01606g on Y. lipolytica phenotype, strain FCY001 and JMY2900 were grown in YNBCasa medium supplemented with glucose or erythritol. Cultures were inoculated at a relatively high biomass (i.e., 0.5 g CDW/ml) and medium was supplemented with casamino acid as energy source for strain FCY001 since this latter has been demonstrated to be unable to grow on YNB-erythritol (FIG. 1A). After 48 h of culture at 28° C., biomasses were equal to 1 and 4 g CDW/ml for strain FCY001 and JMY2900, respectively. Culture supernatants were analyzed by HPLC for the presence of erythritol or erythrulose. For strain JMY2900, erythritol was not detected whereas a residual concentration of 2.6 g/L was measured in culture supernatant of strain FCY001 (data not shown). FIG. 5 shows the UV signals recorded for culture supernatant, pure or mixed with erythrulose or erythritol. For strain FCY001 supernatant, two compounds were eluted at retention time 9.186 and 9.658 min. Based on the chromatogram obtained for supernatants of strains FCY001 and JMY2900 grown on erythrulose and glucose based medium, respectively, these two compounds seems to be related to erythritol catabolism and to be specific of FCY001 mutant. Moreover, addition of pure erythritol in the sample did not modify the elution profile demonstrating that these two compounds do not correspond to erythritol. By contrast, addition of pure erythrulose in the sample, led to an increase of the elution peak intensity of one of the two compounds demonstrating, thus, that it corresponds to erythrulose.

The defect of growth observed for FCY001 in the presence of erythritol together with the detection of erythulose in the culture supernatant of this strain demonstrate clearly that gene YALI0F01606g is involved in erythitol catabolism and that it corresponds to erythritol kinase.

2.7) Erythrulose Production Analysis by NMR

To confirm that the disruption of EYK1 lead to the accumulation of erythrulose, strains FCY001 and wild-type strain W29 were incubated at high cell density in EPF medium for 48 h and, the culture supernatants were analyzed by NMR spectroscopy. For that purpose, EPF medium was inoculated at high cell density (OD 600 nm=2) with Y. lipolytica strains and incubated for 48 h at 250 RPM. Culture supernatants were then used for NMR measurements. Spectra were recorded at 25° C. on a Bruker AVIII HD equipped with a SMART BBFO probe operating at 400 MHz for the ¹H. The pulse sequence used for ¹H detection with water suppression was Perfect-echo Watergate sequence (Adams et al 2013). Spectra were centered on the water signal at 4.7 ppm. 16 transient were added on 32K point during an acquisition time of 2.56 s. The delay for binomial water suppression was 800 μs and the relaxation delay was 1 s. Prior to Fourier transform, data were multiplied with an exponential function to give a broadening of 0.3 Hz. Samples were prepared by mixing 570 μl of Y. lipolytica culture supernatant with 30 μl of D₂O. Erythrulose (Sigma Aldrich) solution at 2 g/L in D₂O was used as a standard.

As shown in FIG. 6, the characteristic signals observed for erythrulose standard solution in the range of 4.32 and 4.54 ppm are clearly present for strain FCY001 as compared to strain W29. This clearly demonstrated that the EYK disrupted strain accumulates erythrulose as compared to the non-disrupted strain.

2.8) The Pull and Push Strategy to Enhance Erythritol Production

### Overexpression of Glycerol Kinase Increase Glycerol Assimilation Rate and Erythritol Productivity

For strain FCY205 (pTEF-GUT/), the specific glycerol consumption rate (q_{GLY}) was increased by 20% as compared to the parental strain [i.e. 0.091 and 0.076 g/(gDCW h), respectively] (Table 3). This increase is in the same range as that obtained for Y. lipolytica strain A101 overexpressing GUT1 (Mironczuk et al 2016).

In strain overexpressing GUT1 (FCY205), erythritol specific productivity (q_{ERY}) was increased by 45% as compared to the wild-type strain [i.e. 0.051 and 0.035 g/(gDCW h), respectively] while yield was increased by a 21% [i.e. 0.56 and 0.46 g/g, respectively].

### Overexpression of Triose Isomerase and Transketolase Leads to an Increase in Erythritol Productivity

Gene encoding TKL1 involved in erythritol synthesis from DHAP, the end product of glycerol catabolism, identified in Y. lipolytica genome as YALI0E06479g, was used to construct strains FCY208.

Strain FCY208 (pTEF-TKL1) also showed a higher conversion yield (Y_{S/P}) as compared to FCY205 (pTEF-GUT1) [i.e. 0.59 and 0.56 g/g, respectively; Table 3]. However, glycerol uptake was found somewhat lower for this mutant (0.068 g·g_{DCW}⁻¹·h⁻¹) as compared to the wild-type strain (0.076 g·g_{DCW}⁻¹·h⁻¹).

Strain FCY205 (pTEE-GUT1) has shown a significant increase in glycerol uptake capacity while strain FCY208 (pTEF-TKL1) was able to convert glycerol into erythritol with the highest yield. To further increase erythritol productivity, these two genes were co-expressed in strain FCY214. In shake flask culture, this strain performed significantly better than JMY2900 in term of erythritol specific productivity (i.e. 65% increase) and cumulates the positive effect observed for strains FCY205 and FCY208, i.e. higher glycerol uptake rate [i.e. 0.095 and 0.091 g/L, respectively] and higher glycerol/erythritol conversion yield [i.e. 0.61 and 0.59 g/L, respectively].

Results are summarized in Table 3 below.

TABLE 3
Dynamic parameters calculated from glycerol uptake and erythritol
synthesis after 8 days of culture in EPF medium for the different
constructed strains

| | Over- | | | | |
|---|---|---|---|---|---|
| | expressed | Biomass | q_{ERY} (g · | q_{GLY} (g · | Y_{S/P} |
| Strain | genes | (g_{DCW}) | g_{DCW}⁻¹ · h⁻¹) | g_{DCW}⁻¹ · h⁻¹) | (g · g⁻¹) |
| JMY2900 | — | 5.30 | 0.035 | 0.076 | 0.46 |
| (WT) | | | | | |
| FCY205 | GUT1 | 4.83 | 0.051 | 0.091 | 0.56 |
| FCY208 | TKL1 | 5.36 | 0.040 | 0.068 | 0.59 |
| FCY214 | GUT1- | 4.81 | 0.058 | 0.095 | 0.61 |
| | TKL1 | | | | |

The values provided are the means of three independent replicates; the standard deviations were less than 10% of the mean. q_{ERY} erythritol specific production rate, q_{GLY} glycerol specific consumption rate, Y_{S/P} glycerol/erythritol conversion yield.

Quantification of the overexpression of gene GUT1 and TKL1 FIG. 8 shows that gene GUT1 and TKL1 are overexpressed in the corresponding strain (ie FCY205, FCY208 and FCY214) between 3 to 16 more than in strain JMY2900.

2.9) Overexpression of Triose Isomerase and Transketolase in Strain RIY203 Further Increases Erythritol Productivity

### Overexpression of the Genes GUT1 and TKL1 was Carried Out in a Strain Wherein the EYK1 Gene (YALI0F01606g) was Disrupted.

Behavior of the resulting strain FCY218 and FCY214 were investigated in bioreactor as compared to strain JMY2900. Results are presented in Table 4 and FIG. 7.

TABLE 4
Results of bioreactor cultures of FCY214 and FCY218.
Standard deviation were less than 10%

| | JMY2900 | FCY214 | FCY218 |
|---|---|---|---|
| Erythritol (g · l⁻¹) | 55.8 | 79.4 | 78.5 |
| Productivity (g · l⁻¹ · h⁻¹) | 0.59 | 0.84 | 1.05 |
| q_{ERY} (g · g_{DCW}⁻¹ · h⁻¹) | 0.046 | 0.057 | 0.071 |
| q_{GLY} (g · g_{DCW}⁻¹ · h⁻¹) | 0.105 | 0.119 | 0.135 |
| Yield (g · g⁻¹) | 0.44 | 0.48 | 0.53 |
| Final biomass (g_{DCW}) | 12.8 | 14.7 | 14.9 |

At the end of the culture of strain FCY214, erythritol concentration in the culture supernatant reached 79.4 g·l⁻¹. That is a significant increase (42%) as compared to the parental strain (55.8 g·l⁻¹). In those conditions, erythritol is produced at a constant rate (0.84 g/L·h) between 24 and 96 h of culture (Table 4).

As expected, the resulting strain FCY218 is unable to reconsume erythritol, especially after glycerol exhaustion in the bioreactor (FIG. 7). As a consequence, strain FCY218 showed a higher q_{GLY} as compared to FCY214 [i.e. 0.135 and 0.119 g/(gDCW h), respectively], a higher erythritol productivity [i.e. 1.05 and 0.84 g/L h⁻¹, respectively] and a higher yield [i.e. 0.53 and 0.48 g/g, respectively] (Table 4). Moreover, the maximal erythritol concentration was obtained in a lag of time reduced by 66%, as compared to strain JMY2900, positively affecting the process profitability.

2.10) Overexpression of YALI0F01650g in a ΔEyk Strain Allows the Conversion of Erythritol into Erythrulose at High Yield

Y. lipolytica gene YALI0F01650g (SEQ ID NO: 7) has 56% identity with gene ODQ69345.1 (SEQ ID NO: 48) and ODQ69163.1 (SEQ ID NO: 49) that encode erythritol dehydrogenase in Lipomyces starkeyi. From this YALI0F01650g was suggested to encode an erythritol dehydrogenase in Y. lipolytica. The disruption of the latter, renamed EYD1, impairs growth on erythritol medium.

Strain RIY210 was constructed by overexpressing YALI0F01650g under the strong constitutive promoter pTEF in strain RIY203. EYD was amplified from JMY2900 genomic DNA by PCR using primers EYD_Surexp_F (SEQ ID NO: 50=GACGGATCCCACAATGGTTTCTTCAGCCGCTACTT) and EYD_surexp_R (SEQ ID NO: 51=GACCCTAGGTTACCAGACGTGGTGGCCAC); designed to introduce a BamHI and AvrII restriction sites in the PCR fragment. The latter was cloned into BamHI/AvrII digested JMP1047 (Lazar et al 2013) vectors and used to transform strain RIY146. The resulting strain RIY210 was then grown in medium YNB containing a mixture of glycerol and erythritol (50/50). Accumulation of erythulose in culture supernatant was estimated by HPLC after 24 h of growth. Results were compared to that obtained for the wild-type strain. As shown in Table 5, erythrulose accumulate in the culture supernatant of strain RIY210. Conversion of erythritol into erythrulose is closed to 65%.

TABLE 5
accumulation of erythrulose in strain W29 and RIY210

| | W29 | RIY210 |
|---|---|---|
| Biomass at t = 0 h (gDCW/L) | 0.58 | 0.58 |
| Biomass at t = 24 h (gDCW/L) | 12.85 | 9.15 |
| Glycerol consumed (g/L) | 10 | 10 |
| Erythritol consumed (g/l) | 10.2 | 7.51 |
| Erythrulose produced (g/L) | 0 | 4.83 |
| Yield (g/g) | 0 | 0.63 |
| Productivity (g/L · h) | 0 | 0.20 |

### CONCLUSIONS

The present invention provides mutant strains impaired in erythritol catabolism with erythritol productivity increased by 72% and a 65% increase in erythritol specific productivity as compared to a wild-type strain, while process duration was reduced by 66%. It also provides a mutant strain impaired in erythritol catabolism with a conversion of erythritol into erythrulose close to 65%. All these advantages were obtained using an inexpensive medium and in a non-optimized process.

### REFERENCES

Altschul, S., et al., 1997. Nucleic Acids Res. 25, 3389-3402.
Barth, G. and Gaillardin, C. 1996. Yarrowia lipolytica. In: Nonconventional Yeasts in Biotechnology. Springer Berlin Heidelberg, Berlin, Heidelberg, pp. 313-388.
Barth, G. and Gaillardin, C. 1997. FEMS microbiology reviews, 19, 219-237.
Blazeck, J., 2011. Applied and Environmental Microbiology. 77: 7905-7914.
Blazeck, J., 2013. Applied Microbiology and Biotechnology. 97:3037-3052.
Blazeck J., et al., 2014. Nature Communications. 5: 3131, DOI: 10.1038/ncomms4131.
Baykov, A. A., et al., 1988. Anal. Biochem. 171, 266-270.
Carly F., et al., 2015. Enhancement of erythritol production from Yarrowia lipolytica by metabolic engineering. In: XVI Congreso Nacional de Biotecnologia y Bioingenieria. 21 al 26 de Junio de 2015, Guadalajara, Jalisco, México.
Dulermo R., Brunel F., Dulermo T., Ledesma-Amaro R., Vion J., Trassaert M., Thomas S., Nicaud J-M. and Leplat C. (2017) Using a vector pool containing variable-strength promoters to optimize protein production in Yarrowia lipolytica. Microb Cell Fact (2017) 16:31. DOI 10.1186/s12934-017-0647-3
Fickers, P., et al., 2003. Journal of Microbiological Methods, 55, 727-737.
Fickers, P., et al., 2005. FEMS Yeast Research, 5, 527-543.
Gaillardin, C., Ribet, A M. 1987. Current Genetics. 11: 369-375.
Ge, C., et al., 2012. Se Pu. 30, 843-846.
Green, M. R., et al., 2012. Molecular cloning: a laboratory manual. Cold Spring Harbor Laboratory Press, Cold Spring Harbor, N.Y., 3 pp.
Gysler, C., et al., 1990, Biotechnology Techniques. 4, 285-290.
Hedfalk, K. 2012 ‘Codon Optimisation for Heterologous Gene Expression in Yeast’. In Springer Protocols: Methods in Molecular Biology. Recombinant protein production in yeast: methods and protocols. Volume 866 pp. 47-55. Springer Eds.
Hellingwerf, F. J., WO2015-147644A1.
Higuchi, R., Krummel, B., and Saiki, R. K. (1988). A general method of in vitro preparation and specific mutagenesis of DNA fragments: study of protein and DNA interactions. Nucleic Acids Res. 16, 7351-7367.
Hirata, Y., et al., 1999, Journal of Bioscience and Bioengineering, 87:630-635.
Ishizuka, H., et al., 1989. Journal of Fermentation and Bioengineering, 68, 310-314.
Ishizuka, H., et al., 1992. Bioscience, Biotechnology, and Biochemistry 56, 941-945.
Ito, H., et al., 1983, Journal of Bacteriology. 153, 163-168.
Jeya, M., et al., 2009. Applied microbiology and biotechnology, 83, 225-231.
Kim, S.-Y., et al., 1997. Biotechnology Letters, 19, 727-729.
Klebe, R. J., et al., 1983, Gene. 25:333-341.
Kuznetsova, E., et al., 2006. The Journal of Biological Chemistry, 281, 36149-36161.
Lazar, Z. et al., 2013, J Ind Microbiol Biotechnol., 40, 1273-1283.
Lee, J.-K., et al., 2000. Biotechnology Letters, 23, 497-500.
Lee, D., et al., Microbial Cell Factories, 2010, 9, 43.
Le Dall, M. T., et al., 1994, Current Genetics. 26:38-44.
Leplat, C., et al., 2015, FEMS Yeast Research, doi: 10.1093/femsyr/fov052.
Lindgren, V., et al., 1977, Journal of Bacteriology, 119, 431-442.
Madzak, C., et al., 2000, Journal of Molecular Microbiology and Biotechnology, 2:207-216.
Madzak, C., et al., 2004, Journal of Biotechnology, 109:63-81.
Maftahi, M., et al., Yeast, 1996, 12:859-868.
Matsushika, A., et al., 2012, Enzyme and Microbial Technology, 51, 16-25.
Miller, G. L., 1959, Anal Chem 31, 426-428.
Mironczuk, A. A., et al., 2014, Journal of Industrial Microbiology and Biotechnology, 41:57-64.
Mizanur R. M., et al., 2001, J. Biosc. Bioeng., 92:237-241.
Moon, H.-J., et al., 2010. Applied Microbiology and Biotechnology, 86, 1017-1025.
Moonmangnee, D., et al., 2002, Biosci. Biotechnol. Biochem., 66:307-318.
Morii, K., et al., 1985, Anal Biochem., 151, 188-191
Müller, S., et al., 1998. Yeast. 14:1267-1283.
Nicaud, J-M, et al., 2002. FEMS Yeast Research 2/3, 371-379.
Nicaud, J.-M., 2012. Yeast, 29, 409-418.
Nishimura, K., et al., 2006. Journal of Bioscience and Bioengineering; 101:303-308.
Orr-Weaver, T. L., et al., 1981, Proceedings of the National Academy of Sciences of the United States of America, 78:6354-6358.
Querol, A., et al., 1992. Applied and Environmental Microbiology, 58, 2948-2953.
Paradowska and Dagmara (2009). Ann. Universatis Marie-Curie Sklodowska Lublin, 13:47-54.
Ratledge, C. (1994). Yeasts, moulds, algae and bacteria as sources of lipids. Technological advances in improved and alternative sources of lipids. B. S. Kamel, Kakuda, Y. London, Blackie academic and professional, 235-291.
Rymowicz, W., et al., 2008. Biotechnology Letters, 31, 377-380.
Ryu, Y.-W., et al., 2000. Journal of Industrial Microbiology and Biotechnology, 25, 100-103.
Rywińska, A., et al., 2013. Biomass and Bioenergy, 48, 148-166.
Sassi, H., et al 2016, Microbial Cell Factories, 15:159
Song, P., et al., 2011. Fungal Biology, 115, 49-53.
Sprague, G. F., et al., 1977. Journal of Bacteriology, 129, 1335-1342.
Sharma, S., et al., 2012. Plant Signaling and Behaviour, 7, 1337-1345.
Tomaszewska, L., et al., 2012. Journal of Industrial Microbiology & Biotechnology, 39, 1333-1343.
Wang, H. J., et al., 1999, Journal of Biotechnology, 181:5140-5148
Wu, Z. L., 2011, PLoS One, 6, e23172.
Zinjarde, S. S., 2014. Food chemistry, 152, 1-10.

1-18. (canceled)

19. A method for increasing erythritol and/or erythrulose productivity and/or yield of an erythritol and/or erythrulose-producing yeast strain, comprising inhibiting in said yeast strain the expression or the activity of an endogenous L-erythrulose kinase (EC 2.7.1.27) having at least 50% identity with the polypeptide of sequence SEQ ID NO: 1 (YALI_EYK1).

20. The method of claim 19, further comprising overexpressing in said strain at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21), and an invertase (EC 3.2.1.26).

21. The method of claim 19, further comprising overexpressing in said strain an erythritol dehydrogenase (EC 1.1.1.9) and optionally at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21) and an invertase (EC 3.2.1.26).

22. The method of claim 19, wherein erythrulose is not produced, and wherein said method further comprises inhibiting in said strain the expression or the activity of an endogenous erythritol dehydrogenase (EC 1.1.1.9) and optionally overexpressing in said strain at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21) and an invertase (EC 3.2.1.26).

23. The method according to claim 19, wherein the L-erythrulose kinase comprises the consensus amino acid sequence SEQ ID NO: 2.

24. The method according to claim 19, wherein the L-erythrulose kinase has a polypeptide sequence selected from the group consisting of SEQ ID NOS: 1, 3, 4, 5 and 6.

25. The method according to claim 19, wherein the yeast strain belongs to a genus selected from the group consisting of Aurobasidium, Candida, Moniliella, Pseudozyma, Torula, Trichosporon, Trigonopsis and Yarrowia.

26. The method according to claim 25, wherein the yeast strain is selected from the group consisting of Y. lipolytica, Y. galli, Y. yakushimensis, Y. alimentaria and Y. phangnensis.

27. The method according to claim 19, wherein said inhibition is obtained by mutagenesis of an endogenous gene encoding said L-erythrulose kinase.

28. The method according to claim 27, wherein said inhibition is obtained by genetically transforming the yeast strain with a disruption cassette of said endogenous gene.

29. The method according to claim 20, wherein said at least one enzyme is endogenous or from a prokaryotic or eukaryotic organism.

30. The method according to claim 20, wherein the glycerol kinase comprises the amino acid sequence of SEQ ID NO: 8, the glycerol-3P dehydrogenase comprises the amino acid sequence of SEQ ID NO: 9, the triose isomerase comprises the amino acid sequence of SEQ ID NO: 10, the transketolase comprises the amino acid sequence of SEQ ID NO: 11, and the erythrose reductase comprises the amino acid sequence of SEQ ID NO: 12.

31. A method for increasing erythritol productivity and/or yield of an erythritol-producing yeast strain without production of erythrulose, comprising inhibiting in said yeast strain the expression or the activity of an endogenous erythritol dehydrogenase (EC 1.1.1.9) having at least 50% identity with the polypeptide of sequence SEQ ID NO: 7 (YALI_EYD1) and optionally overexpressing in said strain at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21) and an invertase (EC 3.2.1.26).

32. A mutant erythritol and/or erythrulose-producing yeast strain wherein the expression or the activity of an endogenous L-erythrulose kinase is inhibited in the strain, and optionally wherein at least one enzyme selected from the group consisting of an erythritol dehydrogenase (EC 1.1.1.9), a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21), and an invertase (EC 3.2.1.26) is overexpressed in the strain.

33. A mutant erythritol-producing yeast strain that does not produce erythrulose wherein the expression or the activity of an endogenous L-erythrulose kinase and of an endogenous erythritol dehydrogenase is inhibited in the strain, and optionally wherein at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21), and an invertase (EC 3.2.1.26) is overexpressed in the strain.

34. A mutant erythritol-producing yeast strain that does not produce erythrulose, wherein the expression or the activity of an endogenous erythritol dehydrogenase is inhibited in the strain, and optionally at least one enzyme selected from the group consisting of a glycerol kinase (EC 2.7.1.30), a glycerol-3P dehydrogenase (EC 1.1.5.3), a triose isomerase (EC 5.3.1.1), a transketolase (EC 2.2.1.1), an erythrose 4 phosphate phosphatase (EC 3.1.3.23), an erythrose reductase (EC 1.1.1.21), and an invertase (EC 3.2.1.26) is overexpressed in the strain.

35. A method for producing erythritol and/or erythrulose, comprising growing the mutant erythritol and/or erythrulose-producing yeast strain of claim 32 under conditions suitable for production of erythritol and/or erythrulose.

36. A method for producing erythritol, comprising growing the mutant erythritol-producing yeast strain of claim 33 under conditions suitable for production of erythritol.

37. A method for producing erythritol, comprising growing the mutant erythritol-producing yeast strain of claim 34 under conditions suitable for production of erythritol.
